(19) United States
(12) Patent Application Publication
(10) Pub. No.: US 2024/0173292 A1
(43) Pub. Date: May 30, 2024

(54) COMPOUNDS THAT SYNERGIZE WITH COPPER TO KILL STREPTOCOCCUS PNEUMONIAE

(71) Applicant: ARIZONA BOARD OF REGENTS ON BEHALF OF THE UNIVERSITY OF ARIZONA, Tucson, AZ (US)

(72) Inventors: Michael D. L. Johnson, Tucson, AZ (US); Joseph W. Alvin, Tucson, AZ (US); Angela Rivera, Tucson, AZ (US); Wei Wang, Tucson, AZ (US)

(21) Appl. No.: 18/542,376

(22) Filed: Dec. 15, 2023

Related U.S. Application Data
(63) Continuation-in-part of application No. PCT/US22/33805, filed on Jun. 16, 2022.
(60) Provisional application No. 63/211,315, filed on Jun. 16, 2021.
(60) Provisional application No. 63/313,143, filed on Feb. 23, 2022.

(51) Int. Cl. A61K 31/397; A61K 9/00; A61K 31/131; A61K 31/145; A61K 31/426; A61K 31/495; A61P 31/04; A61P 31/10

(52) U.S. Cl. CPC A61K 31/397; A61K 9/0073; A61K 31/131; A61K 31/145; A61K 31/426; A61K 31/495; A61P 31/04; A61P 31/10

(57) ABSTRACT

Copper is broadly toxic to bacteria. The present invention has identified a compound called N,N-dimethyldithiocarbamate (DMDC) and derivatives thereof that combines with copper to prevent the growth of wild type Streptococcus pneumoniae, and even better, kills Streptococcus pneumoniae. Low micromolar levels of DMDC, complexed with biologically relevant amounts of copper, such as those found in the phagolysosome of the macrophage, has a profound effect in killing up to 99.9% of wild type S. pneumoniae in 2 hours. DMDC also works against S. pneumoniae in an animal model of infection, and in vitro against schistosomes and Coccidioides spp. Additionally, the present invention features a method of treating infections caused by the aforementioned pathogenic organisms, as well as others, by administering DMDC to a patient in need thereof.

### CROSS-REFERENCES TO RELATED APPLICATIONS

This application is a continuation-in-part and claims benefit of PCT Application No. PCT/US22/33805 filed Jun. 16, 2022, which claims benefit of U.S. Provisional Application No. 63/211,315 filed Jun. 16, 2021 and U.S. Provisional Application No. 63/313,143 filed Feb. 23, 2022, the specifications of which are incorporated herein in their entirety by reference.

### STATEMENT REGARDING FEDERALLY SPONSORED RESEARCH OR DEVELOPMENT

This invention was made with government support under Grant No. R35 GM128653 awarded by National Institutes of Health. The government has certain rights in the invention.

### FIELD OF THE INVENTION

The present invention features compositions and methods directed towards treating infections (e.g., lung infections or skin infection) caused by pathogenic organisms.

### BACKGROUND OF THE INVENTION

Microbial resistance to traditional antibiotics is an existential risk and a central focus of global health. Innovation tends to focus on well-studied, canonical targets such as the cell wall (β-lactams) or translation (aminoglycosides). This strong and near-global selection pressure is evident by examples of clinical resistance only years after introduction. By iteratively selecting a single target or cell function, a pathogen may only need a few mutations to escape. Therefore, new methods and therapies to target microbes are needed.

Streptococcus pneumoniae (the pneumococcus) is a causative agent of pneumonia, otitis media, meningitis, and sepsis. When grown aerobically, the pneumococcus uses pyruvate oxidase to generate acetyl phosphate, which also produces hydrogen peroxide (H₂O₂). S. pneumoniae does not produce a catalase, which might suggest this bacterium is more sensitive to H₂O₂ stress. However, S. pneumoniae survives exposure to 10 mM H₂O₂, and produces large amounts of peroxide (˜100 μM·h-1; [H₂O₂]max>1 mM). Considering these conditions, S. pneumoniae is remarkably resistant to Cu²⁺ in standard media, overcoming concentrations above 2 mM. This resistance and the importance of copper export in pneumococcal colonization and persistence make this organism an appealing model to study aspects of copper toxicity as a way to develop new therapeutics independent of traditional antibiotics.

### BRIEF SUMMARY OF THE INVENTION

It is an objective of the present invention to provide compositions and methods that allow for the treatment of infections (e.g., lung infections or skin infection) caused by pathogenic organisms (e.g., bacteria or fungi), as specified in the independent claims. Embodiments of the invention are given in the dependent claims. Embodiments of the present invention can be freely combined with each other if they are not mutually exclusive.

The aseptic properties of copper are not completely understood, yet copper toxicity disrupts multiple components of cellular homeostasis. The current model for soluble copper toxicity in vivo is based on its Fenton-like redox activity and displacing native metal cofactors. Within aerobic environments, it's thought that Cu²⁺ is the predominant ion species. Endogenous reducing agents such as glutathione are easily oxidized by Cu²⁺, affording the insoluble and reactive Cu¹⁺ ion and acting to buffer excess copper. Much like Fe²⁺, Cu¹⁺ catalytically reduces H₂O₂ to hydroxide ions and hydroxyl radicals. Through these reactions, exposure to Cu²⁺ can lead to the rapid generation of reactive oxygen species (ROS) and depletion of cellular antioxidants.

The opportunistic pathogen Streptococcus pneumoniae (the pneumococcus) encounters macrophages during initial and protracted infections. The pneumococcus employs a copper export pathway, which improves colonization and persistent infection of the nasopharynx and the upper respiratory tract. Because copper is tightly regulated in the host, The present invention sought to leverage the localized power of nutritional immunity by identifying small molecules with copper-dependent toxicity (CDT). The present invention demonstrates that N,N-dimethyldithiocarbamate (DMDC), and derivatives thereof are copper-dependent antibiotics against S. pneumoniae and have effectiveness against a range of pathogens, from bacteria to fungi to parasites.

The present invention features a composition comprising a N,N-dimethyldithiocarbamate (DMDC):

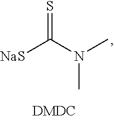

or a derivative thereof as described herein (see Tables 1 and 2).

The present invention features a method of treating an infection caused by a pathogenic organism in a patient in need thereof, the method comprising administering a therapeutic amount of a composition comprising N,N-dimethyldithiocarbamate (DMDC) or derivatives thereof to said patient. The pathogenic organism may be a bacteria (e.g., a gram-positive bacteria), a fungus, or a parasite. In some embodiments, the bacteria is Streptococcus pneumoniae (S. pneumoniae), pneumococcus, or serotypes thereof (see FIG. 29A-29T) or the bacteria is Staphylococcus aureus (S. aureus), or Streptococcus pyogenes (S. pyogenes), Streptococcus anginosus, or Pseudomonas aeruginosa (R aeruginosa).

In some embodiments, the present invention features a method of treating a respiratory infection caused by a pathogenic organism in a patient in need thereof. The method may comprise administering a therapeutic amount of N,N-dimethyldithiocarbamate (DMDC), or a DMDC derivative to said patient. The present invention is not limited to DMDC or derivatives thereof; structurally similar molecules are also encompassed in the present invention. In other embodiments, the present invention features a method of treating a skin infection caused by a pathogenic organism (e.g., Methicillin-resistant Staphylococcus aureus (MRSA) or Staphylococcus epidermidis) in a patient in need thereof, the method comprising administering (e.g., topically) a therapeutic amount of a composition comprising N,N-dimethyldithiocarbamate (DMDC) or derivatives thereof to said patient.

In some embodiments, the present invention features a method of treating a respiratory infection caused by S. pneumoniae in a patient in need thereof. The method may comprise administering a therapeutic amount of DMDC or a DMDC derivative to said patient. In some embodiments, the respiratory infection is pneumonia. In some embodiments, DMDC or the DMDC derivative is administered via inhalation. In further embodiments, the present invention features a method of treating pneumonia caused by S. pneumoniae in a patient in need thereof. In some embodiments, the method comprises administering via inhalation a therapeutic amount of DMDC or a DMDC derivative to said patient.

The present invention also features a composition for use in a method of treating a respiratory infection caused by a pathogenic organism. The composition may comprise DMDC or a derivative thereof. In some embodiments, the present invention features a method of treating a respiratory infection caused by S. pneumoniae. In some embodiments, the composition comprises DMDC or a derivative thereof. In further embodiments, the present invention may feature a composition for a method of treating pneumonia caused by S. pneumoniae. In some embodiments, the composition comprises DMDC or a derivative thereof.

One of the unique and inventive technical features of the present invention is the use of N,N-dimethyldithiocarbamate (DMDC), a compound with copper-dependent toxicity, to treat lung infections caused by pathogenic organisms (e.g., S. pneumoniae). Without wishing to limit the invention to any theory or mechanism, it is believed that the technical feature of the present invention advantageously provides for the ability to fight pathogens independent of their antibiotic-resistant status. In vitro, low micromolar levels of DMDC are able to complex with biologically relevant amounts of copper (such as those found in the phagolysosome of the macrophage) and kill up to 99.9% of wild-type Streptococcus pneumoniae in 2 hours. DMDC also works against Streptococcus pneumoniae in an animal model of infection and in vitro against schistosomes and Coccidioides spp. None of the presently known prior references or work has the unique, inventive technical feature of the present invention. For example, prior references do not utilize DMDC complexed with copper to treat respiratory infection (e.g., pneumonia) caused by a pathogenic organism (e.g., bacteria).

Furthermore, the prior references teach away from the present invention. For example, high doses of DMDC can cause neurological problems, and chronic use can harm the liver and lead to higher rates of some carcinomas—including lung adenoma.

Furthermore, the inventive technical features of the present invention contributed to a surprising result. For example, DMDC did not work on Enterococci (a related virulent). This fact was taken advantage of in that it also did not work against the commensal Lactobacillus.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, 1C, and 1D show the metal-binding agent 8HQ and its prochelator form QBP do not cause a significant growth defect to WT TIGR4 but does cause a growth defect to AcopA bacteria. FIG. 1A shows the structure of the metal-binding agent 8HQ. FIG. 1B shows the structure of the prochelator QBP, containing a pinanediol boronic ester masking group to block metal binding prior to protecting group removal by H₂O₂ to produce 8HQ. FIG. 1C shows the percentage of maximal growth of WT TIGR4 or AcopA bacteria under various conditions in ThyB media compared to strain growth with no copper added as measured by maximum optical density OD₆₀₀ (maximal OD₆₀₀˜1.0). Conditions tested included no additions to ThyB media, addition of 1 μM 8HQ, addition of a low level of copper (50 μM for WT and 10 μM ΔcopA), addition of low level of copper+1 μM 8HQ, addition of a higher level of copper leading to half of maximal bacterial growth (500 μM for WT and 50 μM ΔcopA), and addition of a higher level of copper+1 μM 8HQ. FIG. 1D shows the percentage of maximal WT TIGR4 growth under various conditions as measured by maximum optical density OD₆₀₀. Conditions tested included no additions to ThyB media, addition of 1 μM 8HQ, addition of a low level of copper (500 μM for WT and 50 μM ΔcopA), addition of low level of copper+1 μM 8HQ, addition of a higher level of copper leading to half of maximal bacterial growth (50 μM for WT and 10 μM ΔcopA), and addition of a higher level of copper+1 μM 8HQ. All bars represent mean percentage of WT growth±standard error of the mean (SEM) with a minimum of n=12 replicates per condition across 3 independent replicates. Statistical difference measured by Student's t test (*p<0.01, **p<0.001).

FIGS. 2A and 2B show that copper-dependent toxicity (CDT) is not observed for disulfiram (Antabuse, tetraethylthiuram disulfide, TETD) in ThyB. FIG. 2A shows a growth curve of WT TIGR4 exposed to indicated concentrations of copper sulfate and/or TETD. FIG. 2B shows a killing curve of WT TIGR4 exposed to indicated concentrations of copper sulfate and TETD. All bars represent mean±SD with n=3 across 3 independent replicates. Statistical difference measured by Student's t test (****p<0.0001)

FIGS. 3A, 3B, and 3C show copper-dependent toxicity (CDT) is observed for diethyldithiocarbamate (DETDC) for concentrations <100 μM, without bactericidal effect. FIG. 3A shows a growth curve of WT TIGR4 exposed to indicated concentrations of copper sulfate and DETDC. FIG. 3B shows a growth curve of WT TIGR4 exposed to increasing concentrations of DETDC with constant levels of copper of 500 μM. FIG. 3C shows TIGR4 pneumococci were exposed to indicated concentrations of copper sulfate and DETDC. All bars represent mean percentage±SD across 3 independent replicates. Statistical difference measured by student's t test (****p<0.0001).

FIGS. 4A and 4B show growth curves for dimethyldithiocarbamate (DMDC), a compound with robust CDT effect. FIG. 4A shows a growth curve of WT TIGR4 exposed to indicated concentrations of copper sulfate and DMDC. FIG. 4B shows a growth curve of the ΔcopA mutant strain exposed to indicated concentrations of copper sulfate and DMDC. All bars represent mean±SD with n=3 across 4 independent replicates. Statistical difference measured by Student's t test (****p<0.0001)

FIGS. 5A, 5B, 5C, and 5D show killing curves for the bactericidal compound dimethyldithiocarbamate (DMDC). FIG. 5A shows a killing curve for WT TIGR4 bacteria exposed to indicated concentrations of copper sulfate and DMDC, showing viable CFU over time. FIG. 5B shows a killing curve of WT TIGR4 bacteria with increasing concentrations of DMDC with a level of copper set to 500 μM Cu²⁺. FIG. 5C shows a killing curve of WT TIGR4 bacteria with varying concentrations of copper with a level of DMDC set at 32 μM over a 4 hour time period. FIG. 5D shows a killing curve of ΔcopA mutant strain exposed to indicated concentrations of copper sulfate and DMDC. All bars represent mean±SD with n=3 across 3 independent replicates. Statistical difference measured by Student's t test (****p<0.0001).

FIGS. 6A, 6B, 6C, and 6D show that DMDC is an effective antibiotic against a murine Streptococcus pneumoniae infection model. Two groups of five, eight-week-old mice (FIG. 6A, 6B; n=10) or two groups of three 18-month-old mice (FIG. 6C, 6D; n=6) per treatment were infected with bacteria at time 0 and DMDC at either 7 or 14 hours post infection. At 48 hours post infection, animals were sacrificed and (FIG. 6A, 6C) bled, or (FIG. 6B, 6D; n=8) lung titers were measured. Mann-Whitney Wilcoxon ranked-sum tests were used to measure significant differences at p<0.05 and p<0.01. The bar within the data set represents the median.

FIGS. 7A and 7B show a growth curve for dimethyldithiocarbamate (DMDC) against Staphylococcus aureus. FIG. 7A shows a growth curve of S. aureus in BHI media with indicated concentrations of copper sulfate and DMDC. FIG. 7B shows a kill curve of S. aureus exposed to indicated concentrations of copper sulfate and DMDC. All bars for the growth curve represent mean percentage±SEM with a minimum of n=18 replicates per condition across 3 independent replicates. Statistical significance was determined by the Student's t-test (****p<0.0001).

FIGS. 8A and 8B show Coccidioides posadasii displays decreased recovery after exposure to DMDC and copper. CDT was measured in (FIG. 8A) mycelial or (FIG. 8B) spherule killing after incubation in the listed concentrations of DMDC and copper for 48 hours. All bars represent mean percentage±SEM with a minimum of n=3 replicates per condition across 3 independent replicates. Statistical difference measured by student's t test (****p<0.0001).

FIG. 9 shows DMDC in combination with copper, decreases the lung stage Schistosoma mansoni viability. DMDC was added to the indicated concentrations with (red bars) or without (blue bars) 10.0 μM CuSO₄. Bars represent mean percentage viability+SD with n=90 per biological replicate with three independent replicates. Statistical differences were measured using multiple paired t-tests with a two-stage step-up (Benjamini, Krieger, and Yekutieli) and FDR of 1.00% (****p<0.0001, ns=not significant).

FIGS. 10A and 10B show a growth curve for the DMDC derivative, potassium morpholine-4-dithiocarbamate (TLA1) against S. pneumoniae. FIG. 10A shows a growth curve of S. pneumoniae in ThyB media with indicated concentrations of copper sulfate and 32 uL of the DMDC derivative. FIG. 10B shows a growth curve of S. pneumoniae in ThyB media with indicated concentrations of the DMDC derivative and 500 uM of copper sulfate. All bars for the growth curve represent mean percentage±SEM with a minimum of n=18 replicates per condition across 3 independent replicates. Statistical significance determined by the Student's t-test (****p<0.0001)

FIGS. 11A and 11B show a growth curve for the DMDC derivative, dipotassium piperazine-1,4-dicarbodithiate (TLA2), against S. pneumoniae. FIGS. 11A and 11B show a growth curve of S. pneumoniae in ThyB media with indicated concentrations of the DMDC derivative, and 250 uM (FIG. 11A) or 500 uM (FIG. 11B) of copper sulfate. All bars for the growth curve represent mean percentage±SEM with a minimum of n=18 replicates per condition across 3 independent replicates. Statistical significance determined by the Student's t-test (****p<0.0001). Additionally, FIGS. 11A and 11B show a protective effect vs copper at [TLA2]>32 uM.

FIGS. 12A and 12B show a growth curve for the DMDC derivative, sodium 4-(p-tolyl)piperazine-1-carbodithioate (TLA3), against S. pneumoniae. FIG. 12A shows a growth curve of S. pneumoniae in ThyB media with indicated concentrations of copper sulfate and 32 uM of the DMDC derivative. FIG. 12B shows a growth curve of S. pneumoniae in ThyB media with indicated concentrations of the DMDC derivative and 500 uM of copper sulfate. All bars for growth curve represent mean percentage±SEM with a minimum of n=18 replicates per condition across 3 independent replicates. Statistical significance determined by the Student's t-test.

FIGS. 13A and 13B show a growth curve for the DMDC derivative, sodium N-benzyl-N-methyldithiocarbamate (TLA4), against S. pneumoniae. FIG. 13A shows a growth curve of S. pneumoniae in ThyB media with indicated concentrations of copper sulfate and 20 uM of the DMDC derivative. FIG. 13B shows a kill curve of S. pneumoniae exposed to indicated concentrations of copper sulfate and the DMDC derivative. All bars for the growth curve represent mean percentage±SEM with a minimum of n=18 replicates per condition across 3 independent replicates. Statistical significance was determined by the Student's t-test.

FIGS. 14A and 14B show a growth curve for the DMDC derivative, sodium N-allyl-N-methyldithiocarbamate (TLA5), against S. pneumoniae. FIG. 14A shows a growth curve of S. pneumoniae in THyB media with indicated concentrations of copper sulfate and 32 uM of the DMDC derivative. FIG. 14B shows a growth curve of S. pneumoniae in ThyB media with indicated concentrations of the DMDC derivative and 5.00 uM of copper sulfate. All bars for the growth curve represent mean percentage±SEM with a minimum of n=18 replicates per condition across 3 independent replicates. Statistical significance was determined by the Student's t-test (****p<0.0001).

FIGS. 15A and 15B show a growth curve for the DMDC derivative, sodium ((2S,3S)-1-ethoxy-3-methyl-1-oxopentan-2-yl)carbamodithioate (TLA6), against S. pneumoniae. FIG. 15A shows a growth curve of S. pneumoniae in ThyB media with indicated concentrations of copper sulfate and 64 uM of the DMDC derivative. FIG. 15B shows a growth curve of S. pneumoniae in ThyB media with indicated concentrations of the DMDC derivative and 500 uM of copper sulfate. All bars for the growth curve represent mean percentage±SEM with a minimum of n=18 replicates per condition across 3 independent replicates. Statistical significance was determined by the Student's t-test.

FIGS. 16A, 16B, 16C, and 16D show copper-dependent cytotoxicity of DMDC is enhanced in host-niche mimicking media in comparison to nutrient-rich media. FIG. 16A shows a growth curve of WT TIGR4 S. pneumoniae in M17 media supplemented with indicated concentrations of copper and/or DMDC, demonstrating a significant growth defect for the combination of 500 μM Cu²⁺+32 μM DMDC. FIG. 16B shows a killing curve assay of WT TIGR4 starting with an inoculum of 1×10⁷ CFU/mL in M17 media supplemented with indicated concentrations of copper and/or DMDC, demonstrating a significant decrease in CFU/mL to a level below our level of detection at t=120-minutes for the 500 μM Cu²⁺+32 μM DMDC combination, indicating bactericidal activity. FIG. 16C shows a growth curve of WT TIGR4 in RPMI media supplemented with copper and/or DMDC, demonstrating a significant growth defect for the combination of 50 μM Cu²⁺+16 μM DMDC. FIG. 16D shows a killing curve assay of WT TIGR4 starting with an inoculum of 5.8×10⁶ CFU/mL in RPMI media supplemented with copper and/or DMDC, demonstrating a bactericidal combination of 250 μM Cu²⁺+16 μM DMDC at the t=180 and t=240-minutes timepoints. All bars for growth curves represent mean±standard deviation (SD) with n=18 across 3 independent replicates. Statistical difference measured by one-way ANOVA and Dunnett's multiple comparisons test; ns=not significant, *p<0.05, **p<0.01, ***p<0.001, and ****p<0.0001. All bars for killing curves represent mean±standard deviation (SD) with n=9 across 3 independent replicates. Statistical difference was measured by Student's t test; ns=not significant, *p<0.05, **p<0.01, ***p<0.001, and ****p<0.0001.

FIGS. 17A and 17B show DMDC's bactericidal activity requires constant exposure and is temperature dependent. FIG. 17A shows a killing curve of WT TIGR4 S. pneumoniae in M17 media starting with an inoculum of 3.0×10⁶ CFU/mL in M17 media supplemented with indicated concentrations of copper and/or DMDC for 30 minutes before bacteria were pelleted and resuspended in fresh M17 media without supplementation. The killing effect of 500 μM Cu²⁺+32 μM DMDC is sustained as the bacteria show evidence of static CFU count over the next two time points, while the other conditions show recovery of growth between the t=60 minute and t=120 minute time points. FIG. 17B shows a killing curve of WT TIGR4 S. pneumoniae in M17 media performed at 4° C. Starting with an inoculum of 4.4×10⁶ CFU/mL in M17 media supplemented with indicated concentrations of copper and/or DMDC, the killing effect of 500 μM Cu²⁺+32 μM DMDC is ablated as there is no statistically significant difference of CFU counts between the untreated and combined condition at t=240-minutes. All bars for killing curves represent mean±standard deviation (SD) with n=9 across 3 independent replicates. Statistical difference was measured by Student's t test; ns=not significant, *p<0.05, **p<0.01, ***p<0.001, and ****p<0.0001.

FIG. 18 shows DMDC+copper treatment leads to a significant increase in intracellular copper. Graphite Furnace Atomic Absorption Spectroscopy (GFAAS) analysis of bacterial pellets showing a marked statistically significant increase in copper content within the bacterium of 250 μM Cu²⁺+16 μM DMDC-treated bacteria and within the bacterium of 250 μM Cu²⁺+32 μM DMDC-treated bacteria in comparison to the untreated control. Experiments were performed in triplicate with statistical significance of differences was determined through unequal variances t test; ns=not significant, *p<0.05, **p<0.01, ***p<0.001, and ****p<0.0001.

FIGS. 19A and 19B show ICP-OES analysis of DMDC+copper treatment on intra-bacterial zinc, manganese, copper, and calcium levels. FIG. 19A shows ICP-OES analysis of bacterial pellets showing a marked statistically significant increase in copper content within the bacterium of 250 μM Cu²⁺+16 μM DMDC-treated bacteria and within the bacterium of 250 μM Cu²⁺+32 μM DMDC-treated bacteria in comparison to the untreated control. No significant increase was observed for intra-bacterial zinc, manganese, or calcium level for any experimental condition compared to control. FIG. 19B shows mean±standard deviation for the concentrations of each metal for the indicated experimental conditions. Experiments were performed in triplicate with statistical significance of differences was determined through unequal variances t test; ns=not significant, *p<0.05, **p<0.01, ***p<0.001, and ****p<0.0001.

FIG. 20 shows manganese supplementation of DMDC+Cu²⁺-treated S. pneumoniae can rescue toxicity to a threshold amount. The killing curve of WT TIGR4 S. pneumoniae in M17 media starting with an inoculum of 8.0×10⁶ CFU/mL in M17 media supplemented with indicated concentrations of copper and/or DMDC for 30 minutes, at which point all conditions were supplemented with 500 μM Mn²⁺. Manganese supplementation ablated the killing effect of 250 μM Cu²⁺+16 μM DMDC, but was not able to rescue the toxicity of 250 μM Cu²⁺+32 μM DMDC. Rescue is shown by a lack of statistically significant difference in CFU counts at t=60-minutes and t=120-minutes between Untreated and 250 μM Cu²⁺+16 μM DMDC conditions. All bars represent mean±standard deviation (SD) with n=9 across 3 independent replicates. Statistical difference was measured by Student's t test; ns=not significant, *p<0.05, **p<0.01, ***p<0.001, and ****p<0.0001.

FIGS. 21A, 21B, 21C, and 21D show J774A.1 macrophages display enhanced post hoc killing of DMDC+Cu²⁺-treated TIGR4 bacteria. FIG. 21A shows a macrophage killing assay of WT TIGR4 bacteria co-cultured with activated J774A.1 macrophages. Initial inoculum of bacteria given to macrophages was 6.4×10⁸ CFU/mL for an MOI of 10. No statistically significant difference in killing rate or recovered CFU/mL was observed between untreated and 32 μM DMDC-pre-treated macrophages. FIG. 21B shows a macrophage killing assay of WT TIGR4 bacteria as in FIG. 21A with initial inoculum of 9.2×10⁶ CFU/mL for an MOI of 10. No statistically significant difference in killing rate or recovered CFU/mL was observed between untreated and combination-pre-treated macrophages. FIG. 21C shows a macrophage killing assay of WT TIGR4 bacteria co-cultured with activated J774A.1 macrophages given bacteria that were treated with indicated combinations of Cu²⁺ and DMDC. Initial inoculum of bacteria given to macrophages was 7.3×10⁶ CFU/mL (following a 15-minute incubation with indicated conditions) for an MOI of 10. There is a statically significant decrease in recovered CFU/mL between the untreated bacteria and Cu²⁺+DMDC-treated bacteria at t=5-minutes. At this time point, all combination-treated bacteria were cleared by the macrophages, indicating a rapid post hoc bactericidal killing capacity. FIG. 21D shows a macrophage killing assay of WT TIGR4 bacteria as in FIG. 21C with initial inoculum of 9.6×10⁶ CFU/mL for an MOI of 10. No statistically significant difference in post hoc recovery rate was observed between the conditions, however, there was a trend for improved recovery with the macrophage inhibitor treatment conditions. All bars represent mean±standard deviation (SD) with n=9-12 across 3 independent replicates. Statistical difference was measured by Student's t test; ns=not significant; *p<0.05, **p<0.01, ***p<0.001, and ****p<0.0001.

FIGS. 22A, 22B, 22C, 22D, and 22E show mechanisms utilized by the macrophage phagolysosome synergize with DMDC's copper-dependent toxicity. FIG. 22A shows a growth curve of WT TIGR4 S. pneumoniae in M17 media supplemented with indicated concentrations of zinc and/or DMDC, demonstrating a significant growth defect for the combination of 500 μM Zn²⁺+32 μM DMDC. FIG. 22B shows a killing curve assay of WT TIGR4 starting with an inoculum of 1×10⁷ CFU/mL in M17 media supplemented a titration of combinations of zinc±DMDC, showing that the 500 μM Zn²⁺+32 μM DMDC condition is bacteriostatic with no statistically significant difference in CFU/mL for the two compared conditions. FIG. 22C shows a killing curve assay of WT TIGR4 starting with an inoculum of 4.0×10⁶ CFU/mL, S. pneumoniae was incubated in M17 media supplemented with combinations of copper, DMDC, and hydrogen peroxide. Utilizing a lower amount of copper (250 μM compared to 500 μM used in previous figures), lower amount of DMDC (16 μM compared to 32 μM utilized in previous figures), and a moderate amount of hydrogen peroxide (5 mM) to which S. pneumoniae TIGR4 are resistant, the combination of 5 mM H₂O₂+250 μM Cu²⁺+16 μM DMDC displayed robust killing at t=60-minutes that extended into t=120-minutes. FIG. 22D shows a killing curve assay of WT TIGR4 starting with an inoculum of 6.0×10⁶ CFU/mL, S. pneumoniae was incubated in M17 media supplemented with combinations of copper, DMDC, and DPTA NONOate, a nitric oxide-donating compound. The combination of 40 μM DPTA NONOate+250 μM Cu²⁺+16 μM DMDC displayed statistically significant killing at t=60 and t=120. FIG. 22E shows a capsule blot of WT TIGR4 treated with indicated conditions of copper and DMDC, showing a decrease in capsule for the combination-treated condition. All bars for the killing curve represent mean±standard deviation (SD) with n=9 across 3 independent replicates. Statistical difference was measured by Student's t test; ns=not significant, *p<0.05, **p<0.01, ***p<0.001, and ****p<0.0001. Capsule blot is a representative blot of 3 independent replicates.

FIGS. 23A, 23B, 23C, and 23D show the effect of DMDC treatment on macrophage and DC populations in the lung of BALB/c mice infected with TIGR4. Groups of 8-week-old mice were either untreated (none), given DMDC compound (DMDC only), infected with TIGR4 intranasally (TIGR4 only), or treated with DMDC 8 hours post-TIGR4 infection (DMDC+TIGR4). FIG. 23A shows a representative percentage of Ly6G⁺ neutrophils in untreated mice and CD11b⁺CD11c⁻, CD11b⁺CD11c⁺, and CD11b⁻CD11c⁺ cells from CD45⁺ leukocytes of each group. FIG. 23B shows the quantitative percentage of leukocyte populations in FIG. 23A shown as a mean+SEM (n=3 for untreated group, n=10 for all other groups, three assays combined). FIG. 23C shows representative histograms of percentage of F4/80⁻ and F4/80⁺ cells from CD11b⁺CD11c⁺ and CD11b⁻CD11c⁺ populations in FIG. 23A. FIG. 20D shows the quantitative percentage of F4/80⁻ and F4/80⁺ cells in FIG. 23C. Statistical difference was measured by two-tailed, unpaired t-test with Welch's correction; ns=not significant, *p<0.05, **p<0.01, ***p<0.001, and ****p<0.0001.

FIG. 24 shows DMDC treatment is not cytotoxic to J774A.1 macrophages. Using a Trypan blue cytotoxicity assay, incubation of J77A.1 macrophages with up to 32 μM DMDC (the maximum dosage in our assays), did not display a significant difference in cellular viability after a 2-hour incubation. All bars represent mean live cell percentage±standard deviation (SD) with n=3 across 3 independent replicates. Statistical difference was measured by Student's t test; ns=not significant; *p<0.05, **p<0.01, ***p<0.001, and ****p<0.0001.

FIG. 25 shows a Streptococcus pyogenes (M1 GAS) killing curve with DMDC and Cu⁺². All bars represent mean±SD with n=3 across 4 independent replicates. Statistical difference was measured by Student's t test.

FIG. 26 shows a Pseudomonas aeruginosa (PAK strain) killing curve with DMDC and Cu⁺² in MOPS Minimal media. All bars represent mean±SD with n=3 across 4 independent replicates. Statistical difference was measured by Student's t test.

FIG. 27 shows Inductively Coupled Plasma—Optical Emission Spectroscopy (ICP-OES) of Copper plus Other Metals. Values are relative to the addition of copper. The experiment was performed in RPMI. ***P<0.01 relative to plus copper value.

FIG. 28 shows ICP-OES of Copper Inside Iron Import Mutants. There was no significant change in the amount of bacteria or baseline copper across samples #P=0.06 vs. WT.

FIG. 29A-29T shows killing curve data for various S. pnuemoniae clinical isolates (stereotypes). Bars from left to right represent untreated stereotypes, stereotypes treated with 16 uM DMDC, 32 uM DMDC, 250 uM Cu²⁺, 500 uM Cu²⁺, 500 uM Cu²⁺+32 uM DMDC, 500 uM Cu²⁺+16 uM DMDC, 250 uM Cu²⁺+32 uM DMDC, and 250 uM Cu²⁺+16 uM DMDC.

FIG. 30A-30C shows killing curve data for Methicillin-resistant Staphylococcus aureus (MRSA) treated with various DMDC derivatives (e.g., PDBC (FIG. 30A), BMDC (FIG. 30B), AMDC (FIG. 30C)). Bars from left to right represents untreated MRSA, MRSA treated with 16 uM DMDC derivative, 32 uM DMDC derivative, 250 uM Cu²⁺, 500 uM Cu²⁺, 500 uM Cu²⁺+32 uM DMDC derivative, 500 uM Cu²⁺+16 uM DMDC derivative, 250 uM Cu²⁺+32 uM DMDC derivative, and 250 uM Cu²⁺+16 uM DMDC derivative.

FIG. 31A-31C shows killing curve data for Staphylococcus epidermidis treated with various DMDC (FIG. 31A) or derivatives thereof (e.g., BMDC (FIG. 31B), PDBC (FIG. 31C)). Bars from left to right represents untreated S. epidermidis, S. epidermidis treated with 16 uM DMDC or DMDC derivative, 32 uM DMDC or DMDC derivative, 250 uM Cu²⁺, 500 uM Cu²⁺, 500 uM Cu²⁺+32 uM DMDC or DMDC derivative, 500 uM Cu²⁺+16 uM DMDC or DMDC derivative, 250 uM Cu²⁺+32 uM DMDC or DMDC derivative, and 250 uM Cu²⁺+16 uM DMDC or DMDC derivative.

FIGS. 32A and 32B shows killing curve data for Staphylococcus saprophyticus treated with various DMDC (FIG. 31A) or derivatives thereof (e.g., BMDC (FIG. 311B)). Bars from left to right represents untreated S. saprophyticus, S. saprophyticus treated with 16 uM DMDC or DMDC derivative, 32 uM DMDC or DMDC derivative, 250 uM Cu²⁺, 500 uM Cu²⁺, 500 uM Cu²⁺+32 uM DMDC or DMDC derivative, 500 uM Cu²⁺+16 uM DMDC or DMDC derivative, 250 uM Cu²⁺+32 uM DMDC or DMDC derivative, and 250 uM Cu²⁺+16 uM DMDC or DMDC derivative.

### DETAILED DESCRIPTION OF THE INVENTION

Any methods, devices, and materials similar or equivalent to those described herein can be used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the invention described herein.

As used herein, “sodium dimethyldithiocarbamate dihydrate” and “N,N-dimethyldithiocarbamate” may be used interchangeably. Additionally, the aforementioned compounds may be abbreviated “DMDC,” or “SDD,” or “SDDC.” “DMDC” or “SDD” or “SDDC” may be used interchangeably.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. The term does not denote a particular age or sex. Thus, adult and newborn subjects, as well as fetuses, whether male or female, are intended to be included. In one embodiment, the subject is a mammal (e.g., a human) having a disease, disorder or condition described herein. In another embodiment, the subject is a mammal (e.g., a human) at risk of developing a disease, disorder or condition described herein. A “patient” is a subject afflicted with a disease or disorder. The term “patient” includes human and veterinary subjects. In certain instances, the term patient refers to a human.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

The term “effective amount” as used herein refers to the amount of a therapy (e.g., DMDC or derivatives thereof) which is sufficient to reduce and/or ameliorate the severity and/or duration of a given disease, disorder or condition and/or a symptom related thereto. This term also encompasses an amount necessary for the reduction or amelioration of the advancement or progression of a given disease (e.g., lung infections or skin infections), disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of DMDC or derivatives thereof described herein is an amount sufficient to provide a therapeutic benefit in the treatment or management of a lung infection, or to delay or minimize one or more symptoms associated with the lung infection. A therapeutically effective amount of DMDC or derivatives thereof described herein means an amount of therapeutic agent, alone or in combination with other therapies, which provides a therapeutic benefit in the treatment or management of lung infections. The term “therapeutically effective amount” can encompass an amount that improves overall therapy, reduces or avoids symptoms or causes, or enhances the therapeutic efficacy of another therapeutic agent.

The terms “administering”, and “administration” refer to methods of providing a pharmaceutical preparation to a subject. Such methods are well known to those skilled in the art and include, but are not limited to, administering the compositions orally, intranasally, parenterally (e.g., intravenously and subcutaneously), by intramuscular injection, by intraperitoneal injection, intrathecally, transdermally, extracorporeally, mucosally (e.g., nasal, inhalation, pulmonary, sublingual, vaginal, buccal, or rectal), topically or the like.

A composition can also be administered by intranasal administration (intranasally) or administration by inhalant. As used herein, “intranasal administration” means delivery of the compositions into the nose and nasal passages through one or both of the nares and can comprise delivery by a spraying mechanism (device) or droplet mechanism (device), or through aerosolization of the composition. Administration of the compositions by inhalant can be through the nose via delivery by a spraying or droplet mechanism for delivering a composition comprising DMDC, in a pharmaceutically acceptable carrier. The exact amount of the compositions required will vary from subject to subject, depending on the species, age, weight, and general condition of the subject, the severity of the disorder being treated, the particular composition used, its mode of administration, and the like. Thus, it is not possible to specify an exact amount for every composition. However, an appropriate amount can be determined by one of ordinary skill in the art using only routine experimentation given the teachings herein.

As described above, the compositions can be administered to a subject in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Typically, an appropriate amount of a pharmaceutically acceptable salt is used in the formulation to render the formulation isotonic. Examples of the pharmaceutically acceptable carrier include, but are not limited to, saline, Ringer's solution and dextrose solution. The pH of the solution is preferably from about 5 to about 8, and more preferably from about 7 to about 7.5. Further carriers include sustained release preparations such as semi-permeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents, and the like in addition to the compounds disclosed herein.

The pharmaceutical formulation can be administered in a number of ways depending on whether local or systemic treatment is desired, and on the area to be treated. A preferred mode of administration of the composition is via inhalation. Other modes of administration may be orally, topically, or parenterally, for example by intravenous drip, subcutaneous, intraperitoneal, or intramuscular injection. The disclosed compounds can be administered intravenously, intraperitoneally, intramuscularly, subcutaneously, intracavity, or transdermally.

Pharmaceutical compositions for topical or transdermal administration may include ointments, lotions, creams, gels, drops, adherent patches, iontophoresis, suppositories, sprays, liquids, and powders. Conventional pharmaceutical carriers, aqueous, powder or oily bases, thickeners, and the like may be necessary or desirable. A person of skill, monitoring a subject's clinical response, can adjust the frequency of administration and dosage of the medication according to methods known in the art.

As used herein, “copper-dependent toxicity (CDT)” refers to the ability of a compound to have toxicity against a specific organism (i.e., pathogenic organism) in a copper-dependent manner.

As used herein, a “bactericidal compound” refers to a compound that kills bacteria. As used herein, an “antimicrobial compound” refers to a compound that destroys or inhibits the growth of microorganisms and especially pathogenic microorganisms. In some embodiments, as used herein, an “antimicrobial compound” may refer to a compound that destroys pathogenic organisms (e.g., fungi).

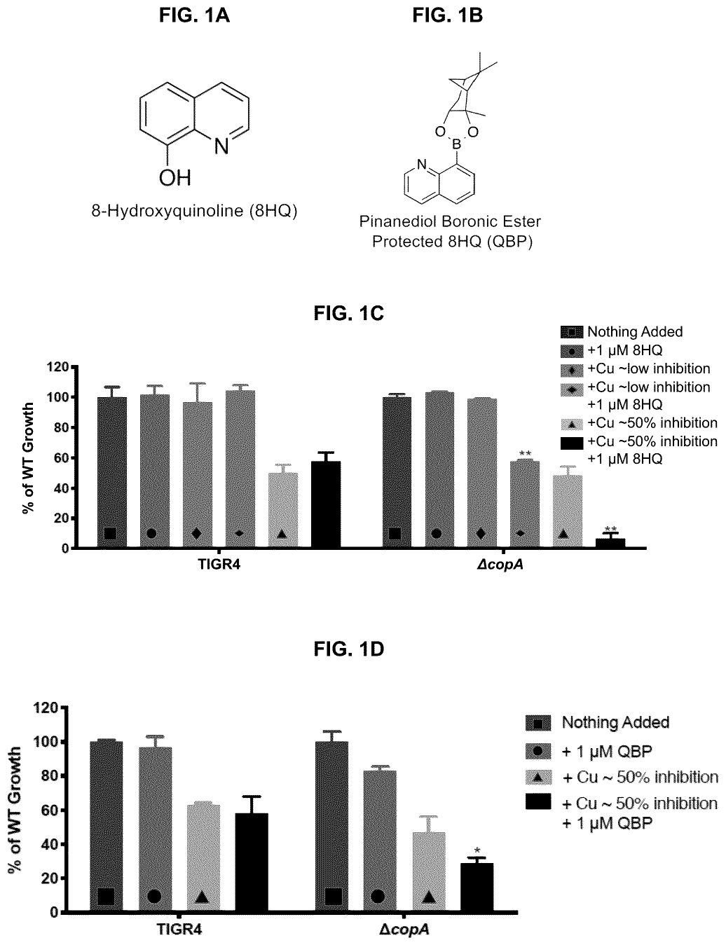

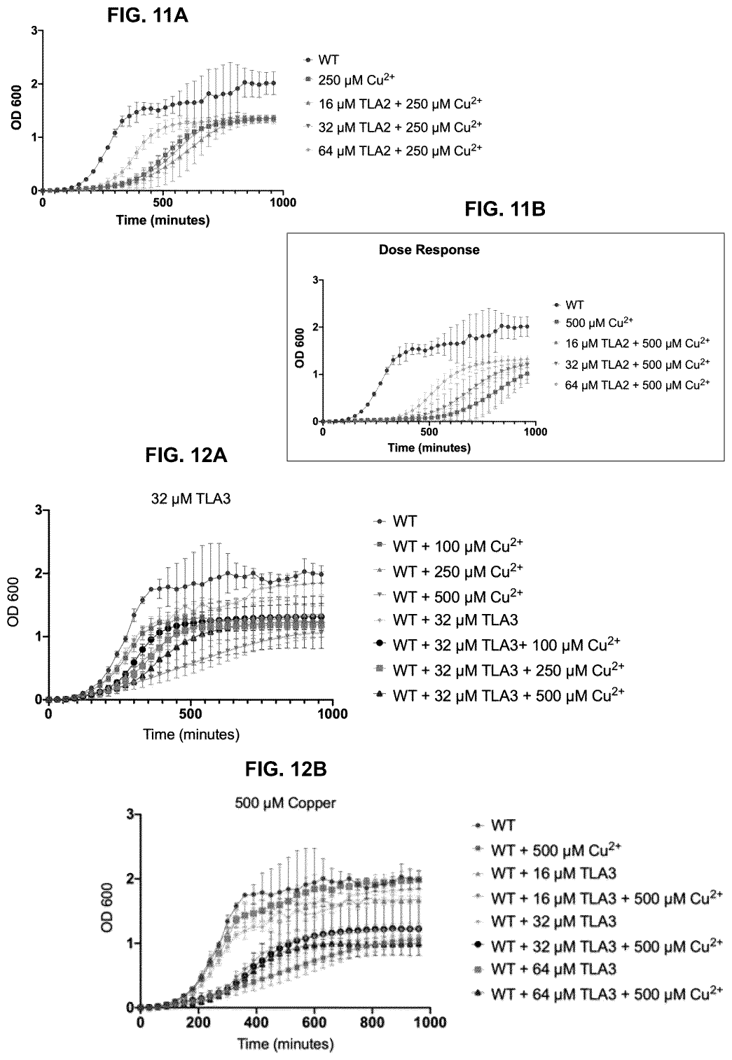

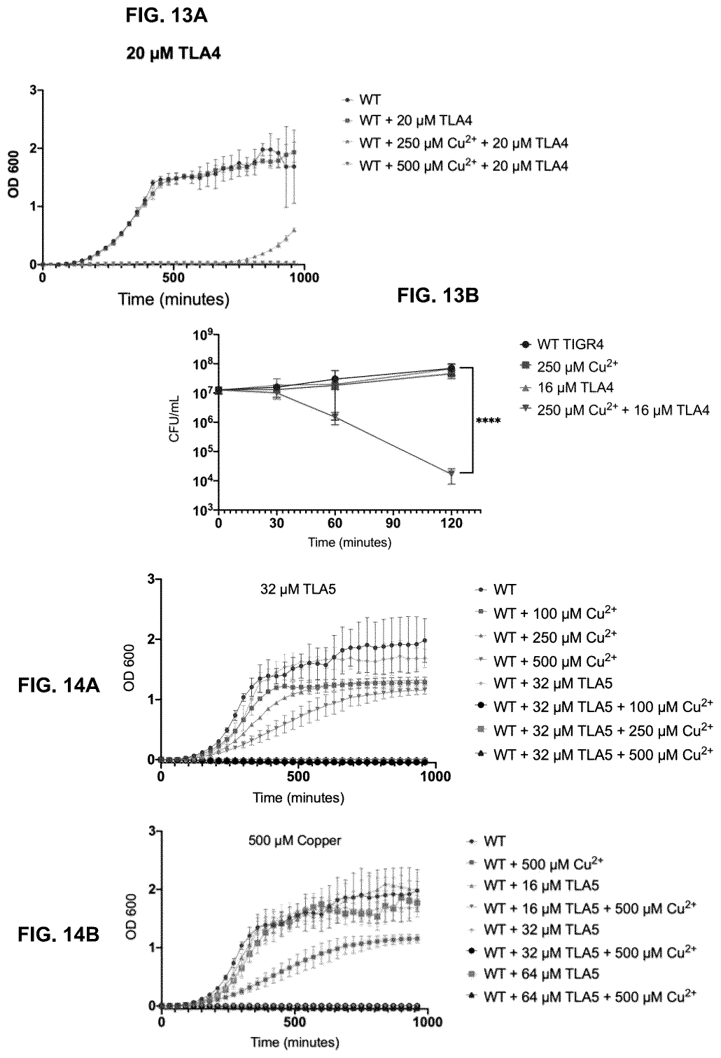

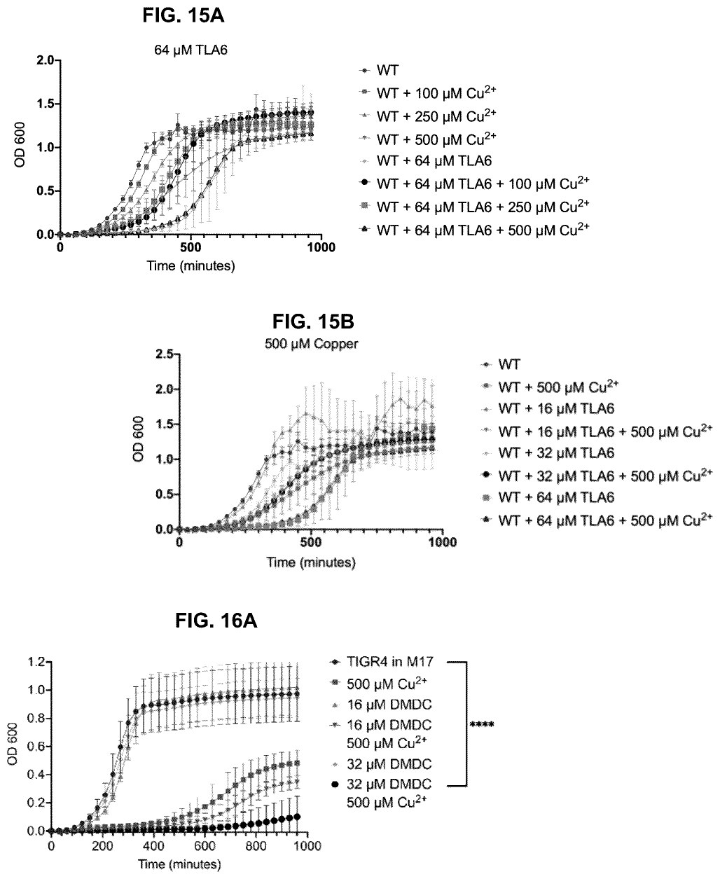

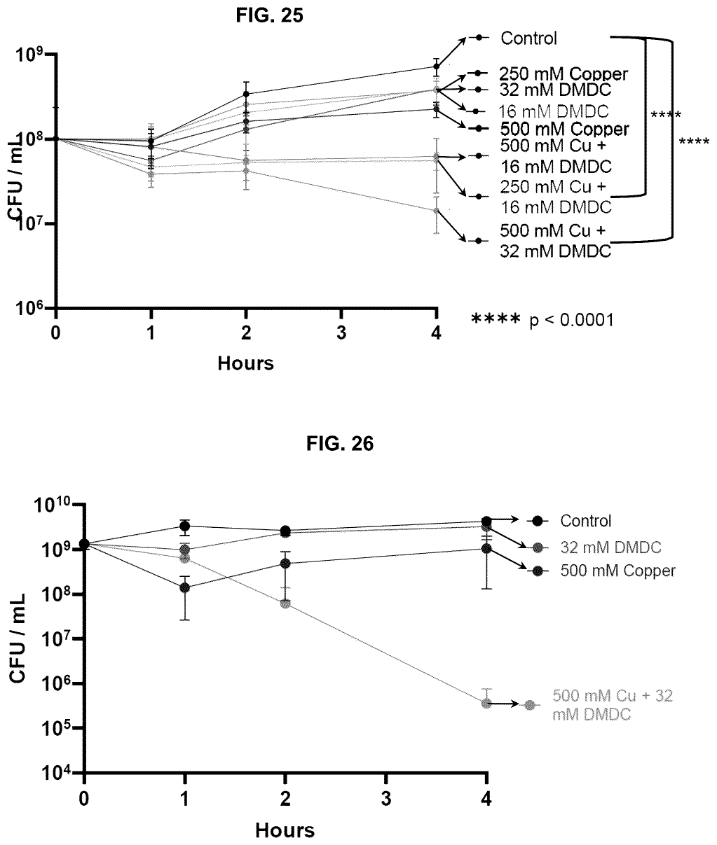

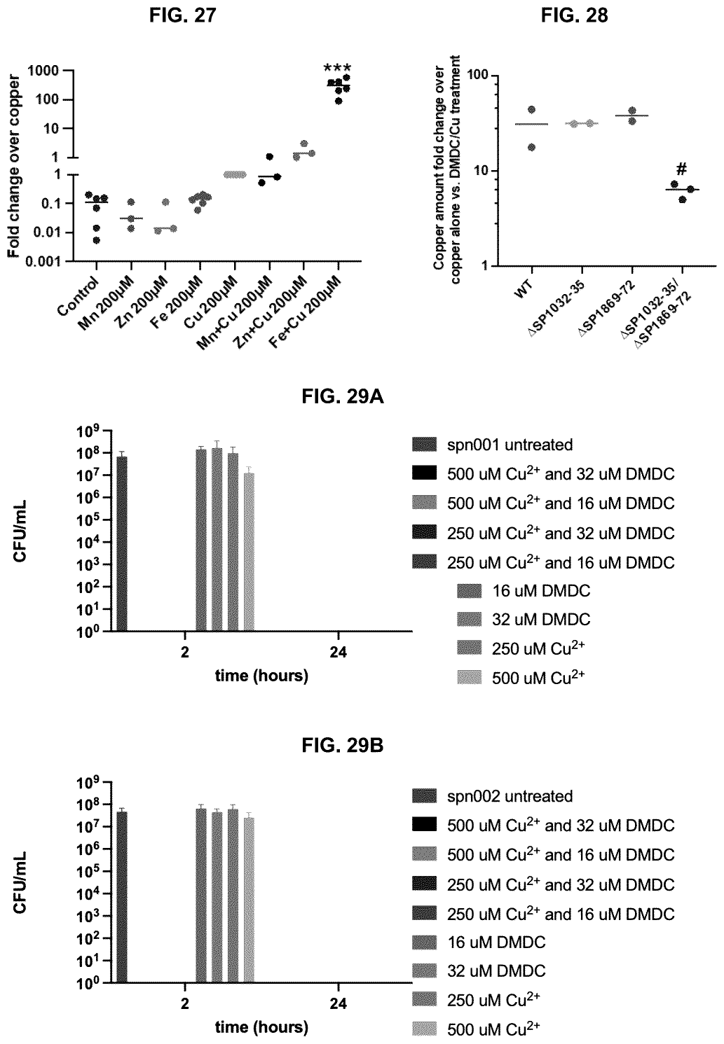

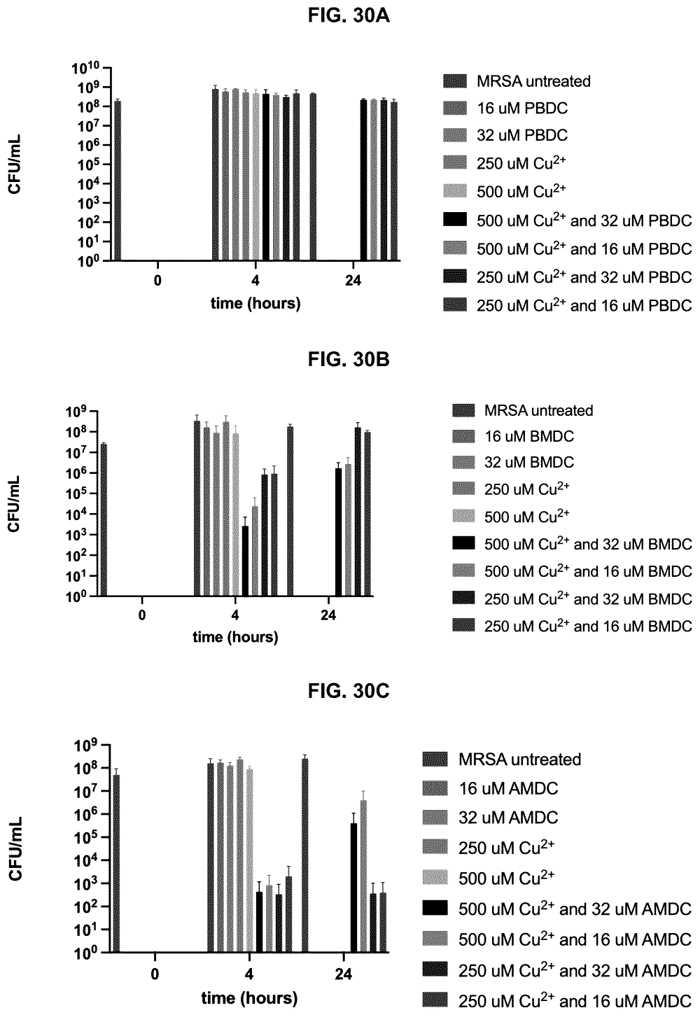

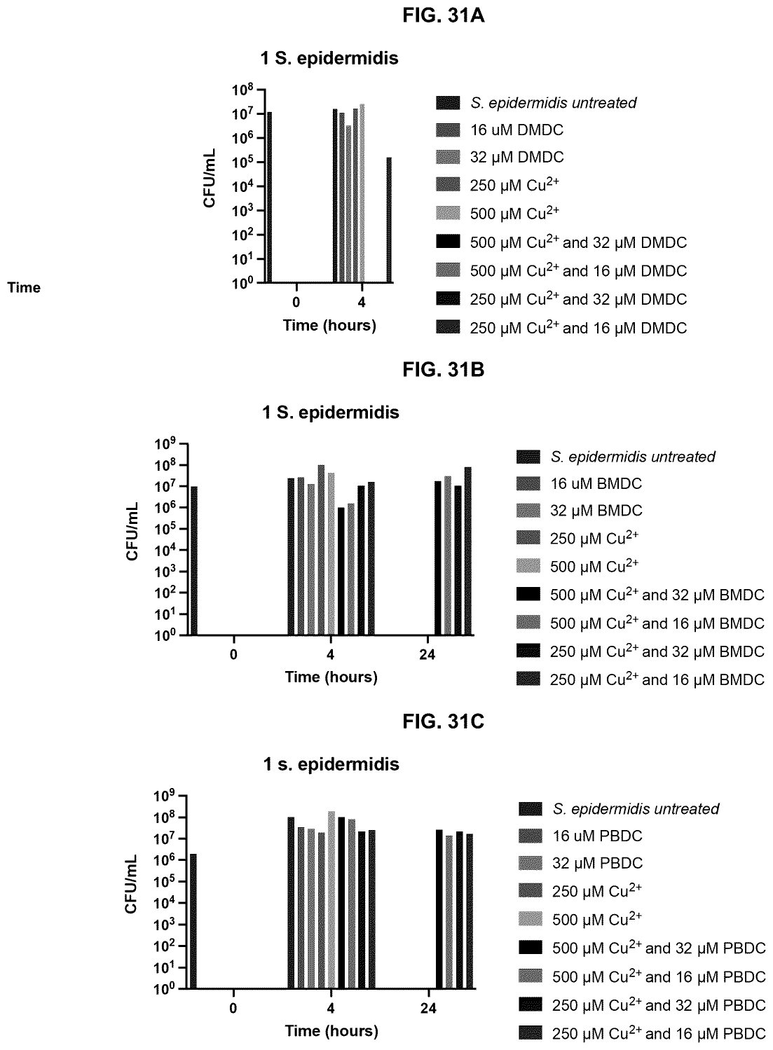

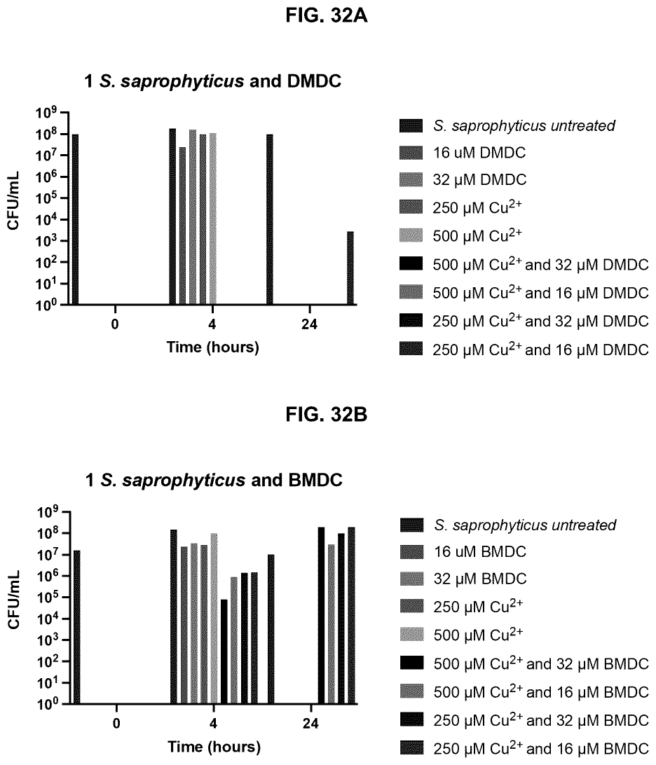

Referring now to FIGS. 1A-32B, the present invention features methods and compositions for the treatment of a lung infection in a subject in need thereof.

### Compositions:

The present invention features a composition comprising N,N-dimethyldithiocarbamate (DMDC);

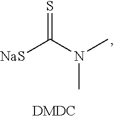

or a derivative thereof as described herein:

Table 1: Shows non-limiting examples of DMDC derivative/analogs described herein.

| Hit Compound (DMDC): | 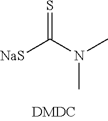 | |
|---|---|---|
| DMDC Acyclic Analogues 1: | DMDC Cyclic Analogues 2: | DMDC Cyclic Analogues 3: |
| 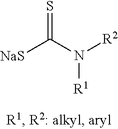 | 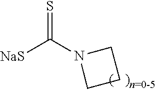 | 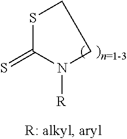 |

In some embodiments, the DMDC derivative may comprise potassium morpholine-4-dithiocarbamate (TLA1), piperizine bis-dithiocarbamate (TLA2, PMCB), sodium 4-(p-tolyl)piperazine-1-carbodithioate (TLA3), sodium N-benzyl-N-methyldithiocarbamate (TLA4, BMDC), sodium N-allyl-N-methyldithiocarbamate (TLA5, AMDC), sodium methyl(2-methylallyl)carbamodithioate (TLA5-1), sodium dial lylcarbamodithioate (TLA5-2), sodium allyl(benzyl)carbamodithioate (TLA5-3), sodium 2,5-dihydro-1H-pyrrole-1-carbodithioate (TLA5-4), sodium 3,6-dihydropyridine-1(2H)-carbodithioate (TLA5-5), sodium ((2S,3S)-1-ethoxy-3-methyl-1-oxopentan-2-yl)carbamodithioate (TLA6). The present invention is not limited to DMDC and derivatives thereof; structurally similar molecules are also encompassed in the present invention.

Table 2: Shows non-limiting DMDC derivatives/analogs described herein. In some embodiments, the derivative of DMDC is according to one of the following:

| | |
|---|---|
| 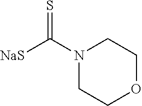 | 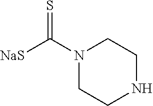 |
| 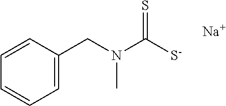 | 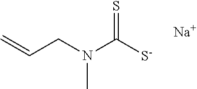 |
| 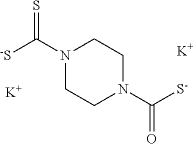 | 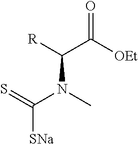 |
| 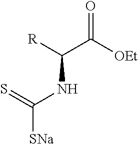 | 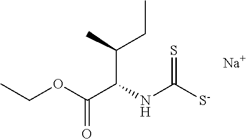 |
| 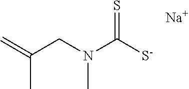 | 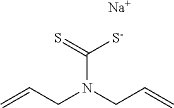 |
| 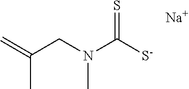 | 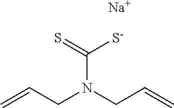 |
| 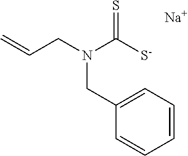 | 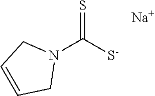 |
| 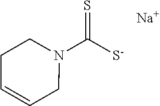 | 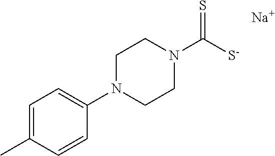 [SMILES: Cc1ccc(N2CCN(C(=S)[S-])CC2)cc1 ; [Na+]] |

In some embodiments, the derivative of DMDC is according to one of the following:

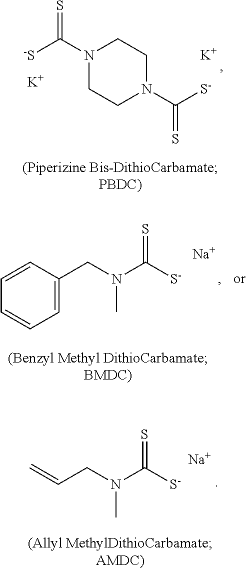

### Methods of Use

The present invention features a method of treating an infection caused by a pathogenic organism in a patient in need thereof, the method comprising administering a therapeutic amount of a composition comprising N,N-dimethyldithiocarbamate (DMDC) or derivatives thereof to said patient. In some embodiments, the infection is a respiratory infection. In other embodiments, the infection is a skin infection. In some embodiments, the infection is a urinary tract infection (UTI). In other embodiments, the infection is vaginosis. In further embodiments, the infection is tooth decay or oral dysbiosis.

In some embodiments, the present invention features a method of treating a respiratory infection caused by a pathogenic organism (e.g., Streptococcus pneumoniae (S. pneumoniae), pneumococcus, or serotypes thereof) in a patient in need thereof, the method comprising administering (e.g., via inhalation) a therapeutic amount of a composition comprising N,N-dimethyldithiocarbamate (DMDC) or derivatives thereof to said patient. In other embodiments, the present invention features a method of treating a skin infection caused by a pathogenic organism (e.g., Methicillin-resistant Staphylococcus aureus (MRSA) or Staphylococcus epidermidis) in a patient in need thereof, the method comprising administering (e.g., topically) a therapeutic amount of a composition comprising N,N-dimethyldithiocarbamate (DMDC) or derivatives thereof to said patient.

In some embodiments, the present invention may feature a method of treating a urinary tract infection caused by a pathogenic organism (e.g., Staphylococcus saprophyticus). The method may include administering a therapeutic amount of N,N-dimethyldithiocarbamate (DMDC) or a DMDC derivative to said patient. In other embodiments, the present invention may feature a method of treating tooth decay and/or oral dysbiosis. The method may include administering a therapeutic amount of N,N-dimethyldithiocarbamate (DMDC) or a DMDC derivative to said patient.

In further embodiments, the present invention may feature a method of treating Schistosomiasis caused by a parasite (e.g., a parasitic flatworm, e.g., S. mansoni). The method may include administering (e.g., orally) a therapeutic amount of N,N-dimethyldithiocarbamate (DMDC) or a DMDC derivative to said patient.

In some embodiments, the present invention may also feature a method of treating bacterial vaginosis. The method may comprise administering a therapeutic amount of N,N-dimethyldithiocarbamate (DMDC) or a DMDC derivative to said patient. The present invention is not limited to DMDC, and derivatives thereof or structurally similar molecules are also encompassed in the present invention. Without wishing to limit the present invention to any theory or mechanism it is believed that the compositions described herein are able to treat bacterial vaginosis because Inventors' surprisingly found that the DMDC, and derivatives thereof do not kill Lactobacillus (known to be decreased in bacterial vaginosis) but does kill bacteria (e.g., anaerobic bacteria) that cause the disease. In some embodiments, the composition is administered vaginally (e.g., topically or mucosally).

The present invention features a method of treating a respiratory infection caused by a pathogenic organism in a patient in need thereof. The method may comprise administering a therapeutic amount of N,N-dimethyldithiocarbamate (DMDC) or a DMDC derivative to said patient. The present invention is not limited to DMDC, and derivatives thereof or structurally similar molecules are also encompassed in the present invention.

In some embodiments, the present invention features a method of treating a respiratory infection caused by S. pneumoniae in a patient in need thereof. The method may comprise administering a therapeutic amount of DMDC or a DMDC derivative to said patient. In some embodiments, DMDC or the DMDC derivative is administered via inhalation. In further embodiments, the present invention features a method of treating pneumonia caused by S. pneumoniae in a patient in need thereof. In some embodiments, the method comprises administering via inhalation a therapeutic amount of DMDC or a DMDC derivative to said patient.

In some embodiments, the DMDC derivative may comprise potassium morpholine-4-dithiocarbamate (TLA1), piperizine bis-dithiocarbamate (TLA2, PMCB), sodium 4-(p-tolyl)piperazine-1-carbodithioate (TLA3), sodium N-benzyl-N-methyldithiocarbamate (TLA4, BMDC), sodium N-allyl-N-methyldithiocarbamate (TLA5, AMDC), sodium methyl(2-methylallyl)carbamodithioate (TLA5-1), sodium diallylcarbamodithioate (TLA5-2), sodium allyl(benzyl)carbamodithioate (TLA5-3), sodium 2,5-dihydro-1H-pyrrole-1-carbodithioate (TLA5-4), sodium 3,6-dihydropyridine-1(2H)-carbodithioate (TLA5-5), sodium ((2S,3S)-1-ethoxy-3-methyl-1-oxopentan-2-yl)carbamodithioate (TLA6). The present invention is not limited to DMDC and derivatives thereof; structurally similar molecules are also encompassed in the present invention. For example, compounds (e.g., DMDC and derivatives thereof) described herein look similar to siderophores (e.g., iron scavenging molecules), that have copper instead of iron. Thus, the bacteria (e.g., the pathogenic organism) uptake the compounds expecting iron but get intoxicated by copper. Thus, compositions comprising compounds similar to siderophores may be used in accordance with the present invention.

As used herein, a “pathogenic organism” refers to an organism capable of causing disease in its host and may also refer to organisms with antimicrobial resistance. In some embodiments, the pathogenic organism is a bacteria (e.g., a Gram positive bacteria). In some embodiments, the pathogenic organism is a fungus. In some embodiments, the pathogenic organism is a parasite. In other embodiments, the pathogenic organism is a parasitic flatworm.

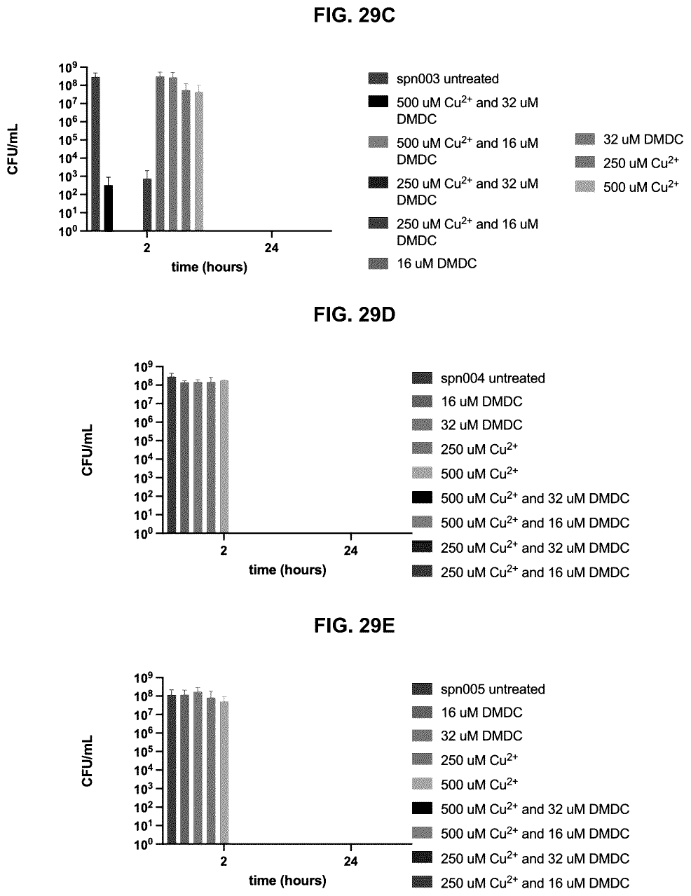

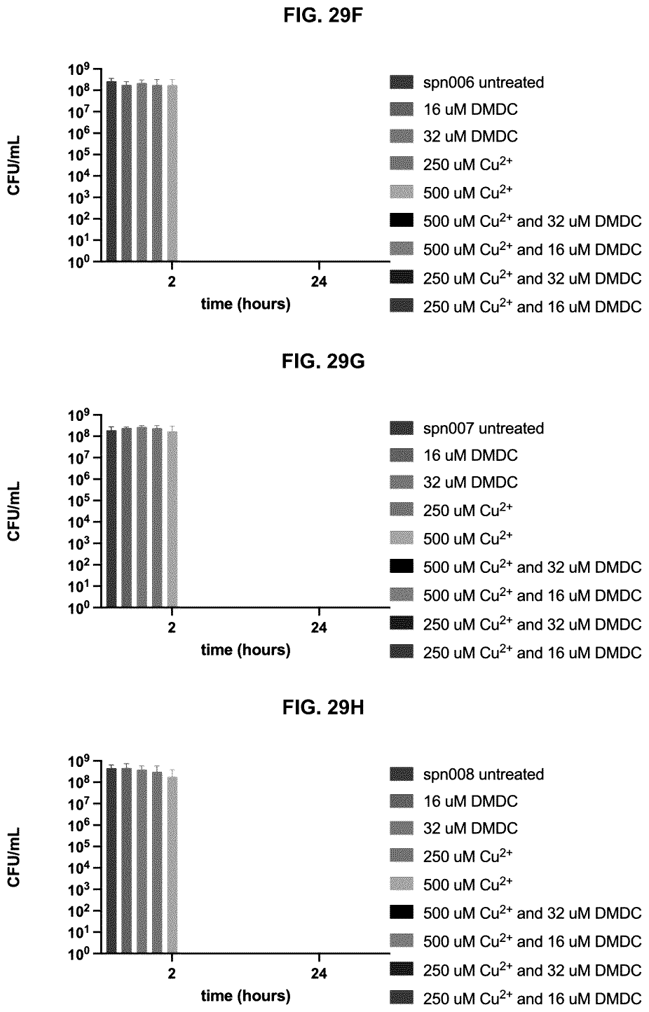

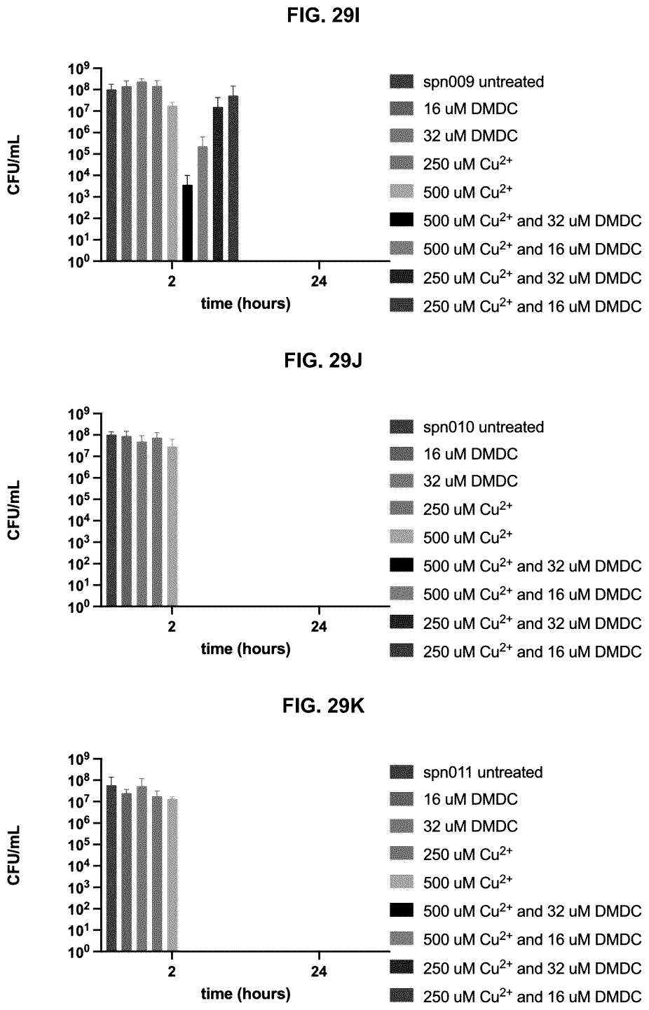

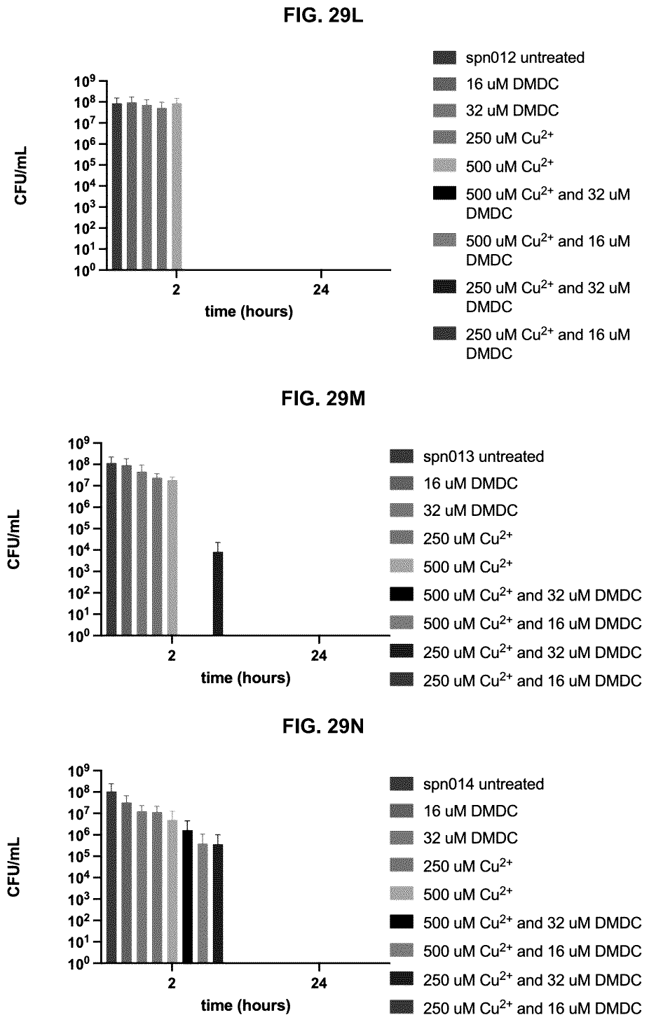

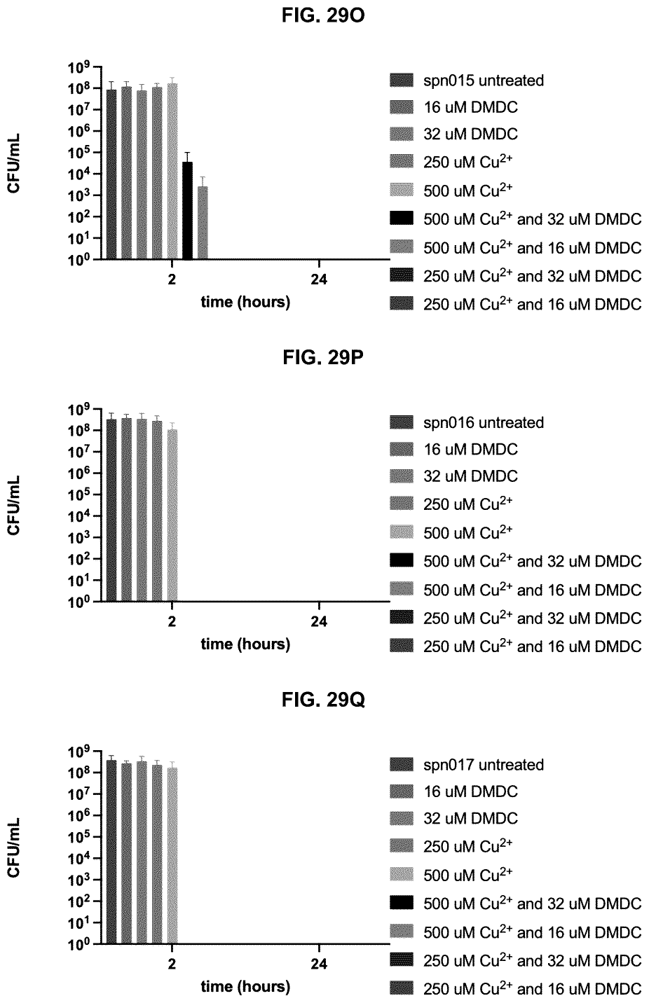

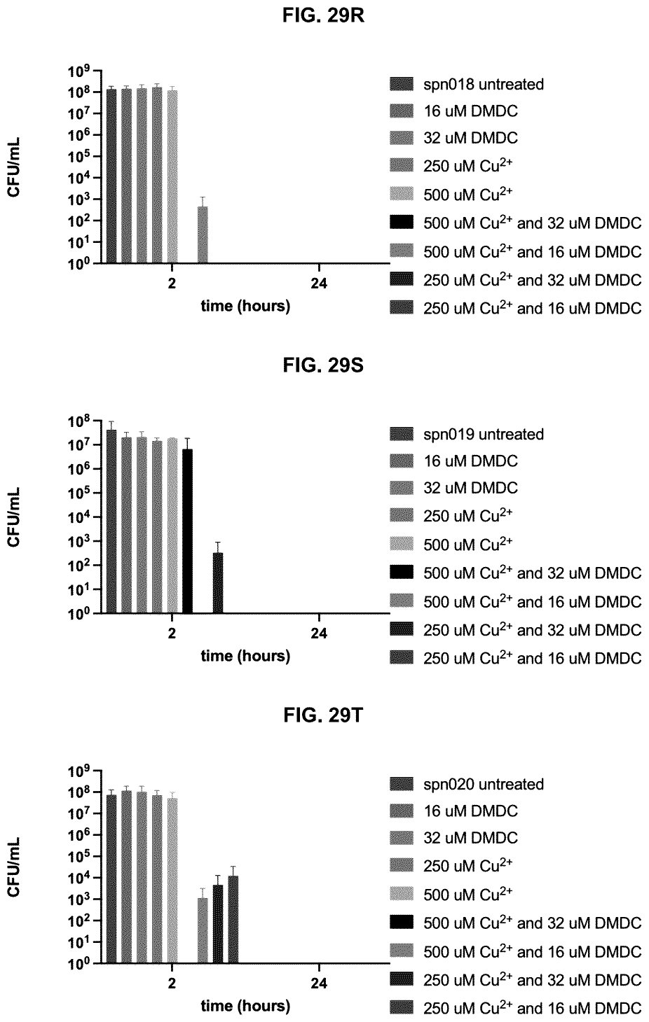

In some embodiments, the bacteria is Streptococcus pneumoniae (S. pneumoniae), pneumococcus, or serotypes thereof (see FIG. 29A-29T). In other embodiments, the bacteria is Staphylococcus aureus (S. aureus), or Streptococcus pyogenes (S. pyogenes), Streptococcus anginosus, or Pseudomonas aeruginosa (R aeruginosa). In further embodiments, the bacteria may include any Streptococcus species, including but not limited to S. pneumoniae, S. pyogenes, S. anginosus, S. agalactiae, S. gallolyticus, S. anginosus, S. sanguinis, S. mitis, or S. mutans. In other embodiments, the bacteria may include any Staphylococcus species, including but not limited to Methicillin-resistant Staphylococcus aureus (MRSA), Staphylococcus epidermidis, or Staphylococcus saprophyticus. In some embodiments, the fungus is Coccidioides posadasii (C. posadasii). In some embodiments, the parasitic flatworm is Schistosoma mansoni (S. mansoni).

Table 5: Shows non-limiting examples of the progression of infections caused by various pathogenic organisms and administration routes that can be employed to optimize therapeutic outcomes using compositions comprising DMDC or derivatives.

| microbe | | |
|---|---|---|
| (e.g., pathogenic organism) | disease progression | administration route |
| Coccidioides (Valley Fever) | respiratory | inhalation |
| Streptococcus pyogenes | skin, respiratory | topically or inhalation |
| (group A strep) | | |
| Streptococcus anginosus | mucous membrane | topically |
| (bacterial vaginosis) | | |
| Streptococcus agalactiae | mucous membrane | topically |
| (group B strep) | and skin | |
| Streptococcus mutans | mucous membrane | topically |
| (tooth decay) | | |
| Pseudomonas aeruginosa | skin, respiratory | topically or inhalation |
| Methicillin Resistant | skin, respiratory | topically or inhalation |
| Staphylococcus aureus | | |
| Streptococcus pneumoniae | respiratory | inhalation |
| Schistosoma mansoni | respiratory | inhalation |

In some embodiments, the respiratory infection is pneumonia. In other embodiments, the respiratory infection is San Joaquin Valley fever. As used herein, a respiratory infection refers to an infection of a part of the body involved in breathing, such as the sinuses, throat, airways, or lungs. The compositions and derivatives thereof described herein may further be used to treat the infectious cause of a disease or disorder (e.g., a respiratory infection).

In some embodiments, the infection is otitis media. In other embodiments, the infection is meningitis. In some embodiments, the infection is sepsis.

In some embodiments, formulations comprising N,N-dimethyldithiocarbamate (DMDC) or a derivative thereof are administered via inhalation. The dose of DMDC administered depends on where the infection is in the lungs. In other embodiments, formulations comprising N,N-dimethyldithiocarbamate (DMDC) or a derivative thereof are administered topically. In other embodiments, formulations comprising N,N-dimethyldithiocarbamate (DMDC) or a derivative thereof are administered orally, or topically, mucosally, intraperitoneally, or intravenously.

In some embodiments, DMDC or a DMDC derivative complexes with copper. In some embodiments, DMDC or a DMDC derivative complexes with copper in the subject. In other embodiments, DMDC or a DMDC derivative complexes with copper in the lungs of the subject.

In other embodiments, the DMDC or a DMDC derivative is complexed with copper before it is administered to the subject. In some embodiments, a complex of DMDC or a DMDC derivative and copper is administered to the subject. In other embodiments, formulations comprising DMDC or a DMDC derivative are complexed with copper before being administered to the subject. In some embodiments, formulations comprising a complex of copper and DMDC or a DMDC derivative are administered to the subject.

In some embodiments, the dose of DMDC or a DMDC derivative administered ranges from about 5 mg to 50 mg, or about 50 mg to 100 mg, or about, 100 mg to 150 mg, or about 150 mg to 200 mg, 200 mg to 250 mg, or about 250 mg to 300 mg, or about 300 mg to 350 mg, or about 350 mg to 400 mg, or about 400 mg to 450 mg, or about 450 mg to 500 mg, 500 mg to 600 mg, or about 600 mg to 700 mg, or about 700 mg to 800 mg, or about 800 mg to 900 mg, or about 900 mg to 1000 mg. Dosage can vary and can be administered in one or more doses daily, for one or several days or weeks (e.g., 7-14 days). Guidance can be found in the literature for appropriate dosages for given classes of pharmaceutical products.

The present invention also features a composition for use in a method of treating a respiratory infection caused by a pathogenic organism. In some embodiments, the composition comprises DMDC or a DMDC derivative. In some embodiments, the present invention features compositions for use in a method of treating a respiratory infection caused by S. pneumoniae. In some embodiments, the composition comprises DMDC or a DMDC derivative. In further embodiments, the present invention may feature a composition for use in a method of treating pneumonia caused by S. pneumoniae. In some embodiments, the composition comprises DMDC or a DMDC derivative.

The present invention features a method of treating a respiratory infection caused by a pathogenic organism in a patient in need thereof, the method comprising administering a therapeutic amount of a derivative of DMDC as described herein.

In some embodiments, formulations comprising DMDC or DMDC derivatives as described herein may be administered via inhalation. In some embodiments, DMDC or derivatives thereof as described herein complexes with copper. In some embodiments, DMDC or derivatives thereof as described herein complexes with copper in the subject. In other embodiments, DMDC or derivatives thereof as described herein complexes with copper in the lungs of the subject. In other embodiments, formulations comprising the DMDC or derivatives thereof as described herein are complexed with copper before being administered to the subject. In some embodiments, formulations comprising a complex of copper and DMDC or derivatives thereof as described herein are administered to the subject.

The present invention also features a method of treating a respiratory infection caused by S. pneumoniae in a patient in need thereof, the method comprising: administering a therapeutic amount of DMDC or DMDC derivative as described herein. In some embodiments, DMDC or DMDC derivative is administered via inhalation.

The present invention further features a method of treating pneumonia caused by S. pneumoniae in a patient in need thereof, the method comprising: administering via inhalation a therapeutic amount of DMDC or derivative thereof as described herein.

In some embodiments, the present invention features a composition for a method of treating a respiratory infection caused by a pathogenic organism, the composition comprising a derivative of DMDC as described herein. In other embodiments, the present invention features a composition for a method of treating a respiratory infection caused by S. pneumoniae, the composition comprising a derivative of DMDC as described herein. In further embodiments, the present invention features a composition for a method of treating pneumonia caused by S. pneumoniae, the composition comprising a derivative of DMDC as described herein

In some embodiments, DMDC derivative complexes with copper in the subject. In other embodiments, DMDC derivative complexes with copper in the lungs of the subject. In other embodiments, the DMDC derivative is complexed with copper before it is administered to the subject. In some embodiments, a complex of a DMDC derivative and copper is administered to the subject.

Formulations may comprise a dosage of the DMDC derivative. In some embodiments, the dose of the DMDC derivative administered ranges from about 5 mg to 50 mg, or about 50 mg to 100 mg, or about 100 mg to 150 mg, or about 150 mg to 200 mg, or about 200 mg to 250 mg, or about 250 mg to 300 mg, or about 300 mg to 350 mg, or about 350 mg to 400 mg, or about 400 mg to 450 mg, or about 450 mg to 500 mg, or about 500 mg to 600 mg, or about 600 mg to 700 mg, or about 700 mg to 800 mg, or about 800 mg to 900 mg, or about 900 mg to 1000 mg. Dosage can vary and can be administered in one or more doses daily, for one or several days or weeks (e.g., 7-14 days). Guidance can be found in the literature for appropriate dosages for given classes of pharmaceutical products.

EXAMPLE 1: The following is a non-limiting example of the present invention. It is to be understood that said example is not intended to limit the present invention in any way. Equivalents or substitutes are within the scope of the present invention.

Bacterial culture: Todd Hewitt Broth+yeast extract (THYB) (BD Biosciences, USA) was prepared according to manufacturer's instructions. Yeast extract was added to a final concentration of 0.2%. Final solution was set to pH 6.6. Tryptic Soy Agar (TSA) (Hardy Diagnostics, USA) was dissolved in deionized water and autoclaved. After cooling the autoclaved TSA, 5% defibrillated sheep's blood (HemoStat Laboratories) of final volume and 20 pg/mL neomycin were added to the solution. These plates (blood agar plates—BAP), were used for routine culture on solid media. Copper stock solutions at 1 M were prepared from CuSO₄ pentahydrate (VWR Life Sciences, USA) in Milli-Q grade water (≥18.0 MΩ cm⁻¹). Colonies from freshly-streaked plates were placed into THYB and grown at 37° C. in 5% CO₂, to an optical density (OD₆₀₀) of 0.13. To prepare working stocks of viable S. pneumoniae, growing cultures are resuspended in fresh media+20% v/v glycerol and stored at −80° C. Aliquot viability and CFU were determined as discussed below before use in experiments. Glycerol stock aliquots were diluted 1:5 into THYB with indicated copper and compound concentrations for assays.

Brain Heart Infusion broth (BHI Media) (Sigma, USA) was prepared following the manufacturer's instructions by dissolving in deionized water and autoclaved. Mannitol Salt Agar (MSA) (MilliPore Sigma, USA) was prepared following manufacturer's instructions. MSA was dissolved in deionized water and autoclaved before pouring into petri dish plates for routine culture on solid media. Staphylococcus aureus (ATCC@ 25923™) was grown at 37° C. in 5% CO₂ to an OD₆₀₀ of 0.13 and diluted 1:5 into BHI for assays. Aliquot viability and density were validated before use in experiments.

Growth Curves: For each small molecule, >97% purity samples were purchased. Each compound was dissolved in DMSO, DMF, ethanol, or water and diluted as needed. Where compounds were water-soluble, dilutions were performed into sterile THYB. Clear 96 well polystyrene plates (Greiner) were arranged to test a range of concentrations from 1 mM diluted fourfold down to 0.24 and a 0 μM control. Frozen aliquots of S. pneumoniae or S. aureus were thawed and diluted five-fold into fresh ThyB or BHI respectively for a total well volume of 200 μL. Assay plates were loaded into a Biotek Cytation5 (Biotek, Vermont, USA) pre-equilibrated to 37° C. and 4% CO₂. Gas control settings were modified for an elevation of 720 m according to manufacturer's directions. The protocol-maintained temperature and CO₂, while measuring absorbance at 600 nm every 30 minutes for 12-16 h.

Killing Curves: Aliquots of S. pneumoniae and S. aureus were thawed and diluted ten-fold into assay conditions prepared in THYB or BHI, respectively. After the indicated incubations at 37° C. in 5% CO₂, samples were serially diluted, plated on BAP or MSA (respectively), incubated overnight at 37° C. in 5% CO₂, and counted to determine viable CFU. Colonies on each plate were counted and multiplied by appropriate dilution factors based on which dilution it was to determine CFU.

Animal experiments: All mouse studies were conducted with prior approval and under the guideline of the Institutional Animal Care and Use Committee at the University of Arizona, IACUC protocol number 18-410, R35 GM128653. All mice were maintained in a biosafety level 2 (BSL2) facility and monitored daily for signs of moribund. Eight-week-old female BALB/cJ mice (Jackson Laboratory) or 18-month-old male and female C57 BL/6 (National Institute on Aging) were anesthetized with 3% isoflurane and intranasally infected with an inoculum of 1×10⁷ CFU viable S. pneumoniae in 25 μL of Tris-Buffered Saline (TBS—50 mM Tris, 150 mM NaCl, pH 7.4). Cohort Controls were given 25 μl TBS. At 7 or 14 hours post infection, mice were treated with doses of intranasal DMDC (0.8 mg/kg or 1.6 mg/kg) in 25 μL TBS. Mice were sacrificed by CO₂ asphyxiation and immediately dissected for lung and blood collection 48 hours post infection. Lung tissue was collected into 1.5 mL tubes, containing 500-μl Phosphate Buffered Saline (DPBS, Gibco), after a brief initial wash in 500-μl PBS to remove any excess blood during dissection. The tissue was then homogenized and centrifuged for 30 seconds at 400 rfc. Blood samples (5-μl volume) were placed in a 45 μl volume PBS solution with heparin (10 UI/mL). Both lung and blood samples were then serially diluted 1:10 and plated on TSA blood plates and incubated overnight at 37° C. and 5% CO₂ for growth. Resulting bacterial colonies were counted for quantification and comparison.

Coccidioides posadasii viability studies: Coccidioides posadasii strain Silveira cultures (Cp) were grown to maturity on 2× glucose-yeast extract (GYE) agar, and arthroconidia (spores) were harvested. Cultures were then incubated with indicated concentrations of CuSO₄ and sodium dimethyldithiocarbamate. The mycelial phase test was performed at the mycelial phase for 48 hrs., 37° C., static. The spherule test was performed at spherule phase for 72 hrs, 38° C., 180 rpm and 20% CO₂ in Modified Converse Media (53, 54). After the respective incubation periods, each sample was diluted 1:100 and plated on GYE to measure viability. The GYE plates were incubated at 37° C. for 4 to 7 days. All manipulation of live fungus was performed at biosafety level 3 with University of Arizona Institutional Biosafety Committee approval.

Juvenile Parasite Collection and Culture: Biomphalaria glabrata snails infected with Schistosoma mansoni (NMRI strain) were obtained from Biomedical Research Institute (BRI; Rockville, MD). Cercariae were shed and mechanical transformation was performed as previously described (55). Newly transformed schistosomula (NTS) were cultured at 37° C. in 5% CO2 using DMEM (Gibco) media supplemented with 10% FBS and 20000 units penicillin and 20 mg streptomycin/mL (2× PenStrep).

DMDC Juvenile Parasite Viability Screening: Approximately 90 schistosomula per well were cultured in a 96-well plate, and each treatment was administered in triplicate. All treatments, including the untreated control samples, were performed in a 200 μL total volume of complete DMEM (Gibco) supplemented with 10% FBS and 2× PenStrep and carried out overnight. Newly transformed schistosomula were treated immediately after transformation-lung stage schistosomula were cultured for 14 days before treatment was administered. Blood supplementation, 2.0 μL concentrated human red blood cells with EDTA, was given after two days of in culture for lung stage schistosomula and repeated every two days. Propidium iodide (PI) at 2 pg/mL was used to stain dead cells within the schistosomula. Fluorescein Diacetate (FDA) at 0.5 pg/mL was used to stain live schistosomula (56). Leica DMI8 fluorescent microscope 10× objective was used to observe and count individuals under brightfield using the Texas red cube set for PI visualization. FITC cube set was used for FDA visualization. Parasites showing any level of PI staining were considered non-viable. Viable individuals were both positive for fluorescein diacetate staining and negative for PI staining. Viability was calculated as follows (FDA positive only/Well Total Population×100=Viability Percentage).

### Results

Human copper levels vary widely with variables of cell type, genotype, time, plus a multitude of environmental factors. In the case of mammalian macrophages, synchrotron x-ray fluorescence (XRF) was used to estimate intra-phagosomal copper concentrations of Mycobacterium tuberculosis-containing vesicles. This XRF study found copper concentration within M. tuberculosis phagosomes widely varies (μ=426±393 μM; n=7) after the first hour of infection. While broad (30-800 μM Cu), the upper limit is roughly 10-20-fold higher than what a bacteria encounters outside of the phagosomal such as in the blood or lungs. Based on these results, concentrations used in this study (240 nM-1 mM) are appropriate for the in vitro experiments described herein.

8-hydroxyquinoline (8HQ) exhibits copper-dependent toxicity (CDT) against various pathogens (FIG. 1A). However, due to the inherent toxicity of 8HQ in mammals, a H₂O₂-labile pinanediol-borate group was added to the hydroxyl group, creating quinoline boronic acid pinanediol ester or QBP (FIG. 1B), to be cleaved in H₂O₂ conditions. However, no 8HQ mediated CDT for WT pneumococcus was seen at several growth inhibitory levels of copper inhibition and thus unsurprisingly, no CDT for QBP as well (FIGS. 1C and 1D). Conversely, a copper exporter deficient mutant (ΔcopA) was susceptible to both QBP and 8HQ at all copper concentrations tested (FIGS. 1C and 1D). The effect of QBP did not exhibit as much CDT on the ΔcopA mutant as compared to 8HQ but it was still a significant reduction (FIGS. 1C and 1D). These data demonstrated that CDT is possible in the pneumococcus, but only under specific conditions and potentially, with different compounds.

Rather than a large-scale approach, 21 compounds were tested with known or predicted copper affinity to evaluate their level of CDT (Table 3, 3). While some compounds were already in complex with metal such as copper phthalocyanine or sodium copper chlorophyllin, others such as penicillamine had been used for clinical metal chelation therapy in patients with Wilson's disease. To test compounds, growth curves were used as an initial screen followed by killing curves. The growth curves were used to test if lag phase bacteria can grow under the constant stress of copper plus compound and if differences from wild type were observed; killing curves were used to examine if any demonstrated CDT from growth curves was bactericidal. Tables, as based on the growth curves, are broken into 1) compounds that had no effect (no effect) (Table 3), 2) compounds that either copper restored growth in the presence of the compound, or, in high concentrations of copper, the compound rescued toxicity in WT or the ΔcopA mutant (protective), 3) compounds that showed a concentration dependent effect of CDT and protection (protective synergistic switch compounds), 4) compounds that showed CDT with just the ΔcopA mutant (mutant synergistic compounds), and 5) compounds that showed synergism against the wild type pneumococcus (WT synergistic) (Table 4).

TABLE 3
Compounds with no effects.

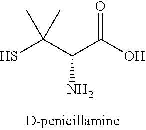

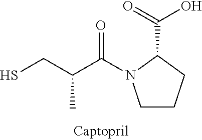

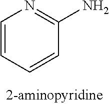

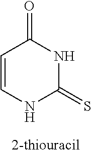

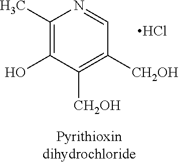

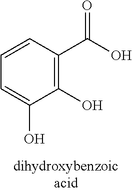 [SMILES: O=C(O)c1cccc(O)c1O]

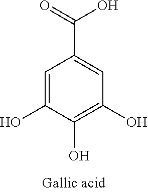 [SMILES: O=C(O)c1cc(O)c(O)c(O)c1]

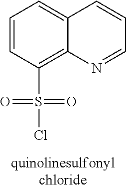 [SMILES: O=S(=O)(Cl)c1cccc2cccnc12]

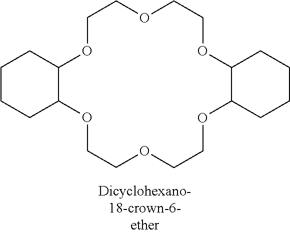 [SMILES: C1CCC2OCCOCCOC3CCCCC3OCCOCCOC2C1]

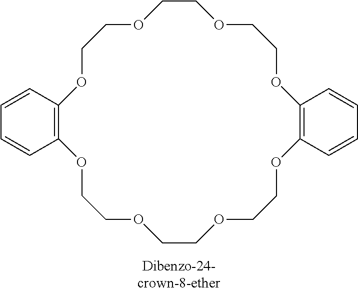 [SMILES: c1ccc2c(c1)OCCOCCOCCOc1ccccc1OCCOCCOCCO2]

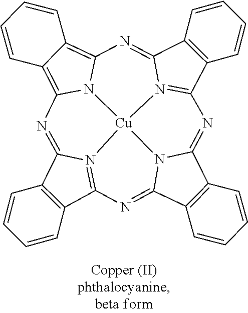

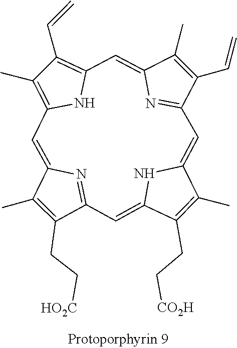 [SMILES: C=CC1=C(C)c2cc3[nH]c(cc4nc(cc5[nH]c(cc1n2)c(C)c5CCC(=O)O)C(CCC(=O)O)=C4C)c(C)c3C=C]

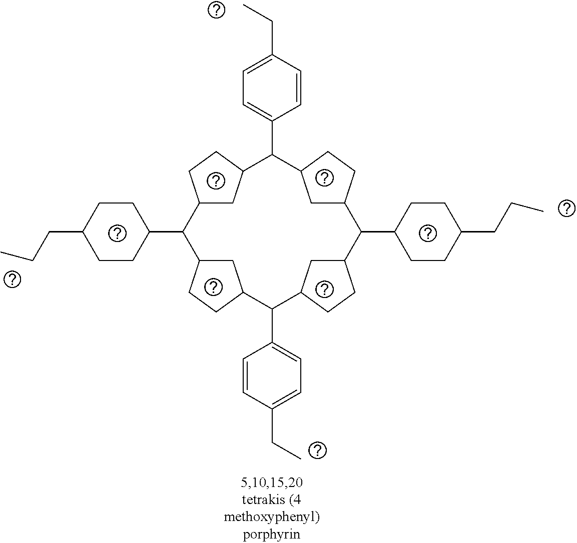 [SMILES: CCCC1CCC(C2C3CCC(C3)C(c3ccc(CC)cc3)C3CCC(C3)C(C3CCC(CCC)CC3)C3CCC(C3)C(c3ccc(CC)cc3)C3CCC2C3)CC1]

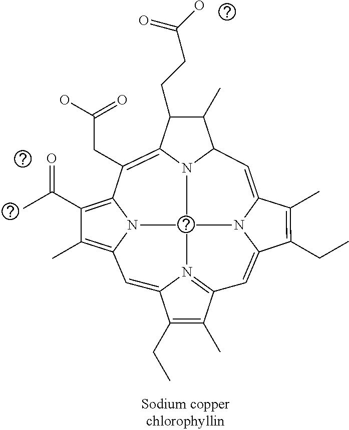

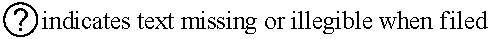

TABLE 4
Effective Compounds

| | | Protective Synergistic Switch | |
|---|---|---|---|
| Protective Compounds | | Compounds | |
| 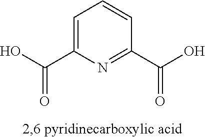 [SMILES: O=C(O)c1cccc(C(=O)O)n1] | 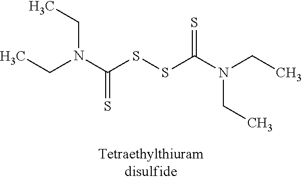 [SMILES: CCN(CC)C(=S)SSC(=S)N(CC)CC] | 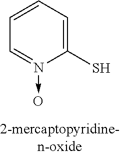 | 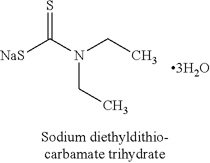 |
| Mutant Synergistic Compounds | | | WT Synergistic |
| 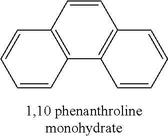 [SMILES: c1ccc2c(c1)ccc1ccccc12] | 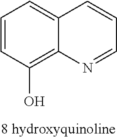 [SMILES: Oc1cccc2cccnc12] | 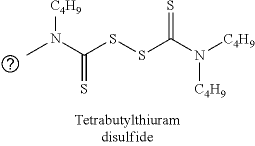 | 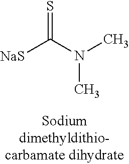 |

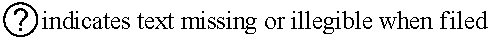

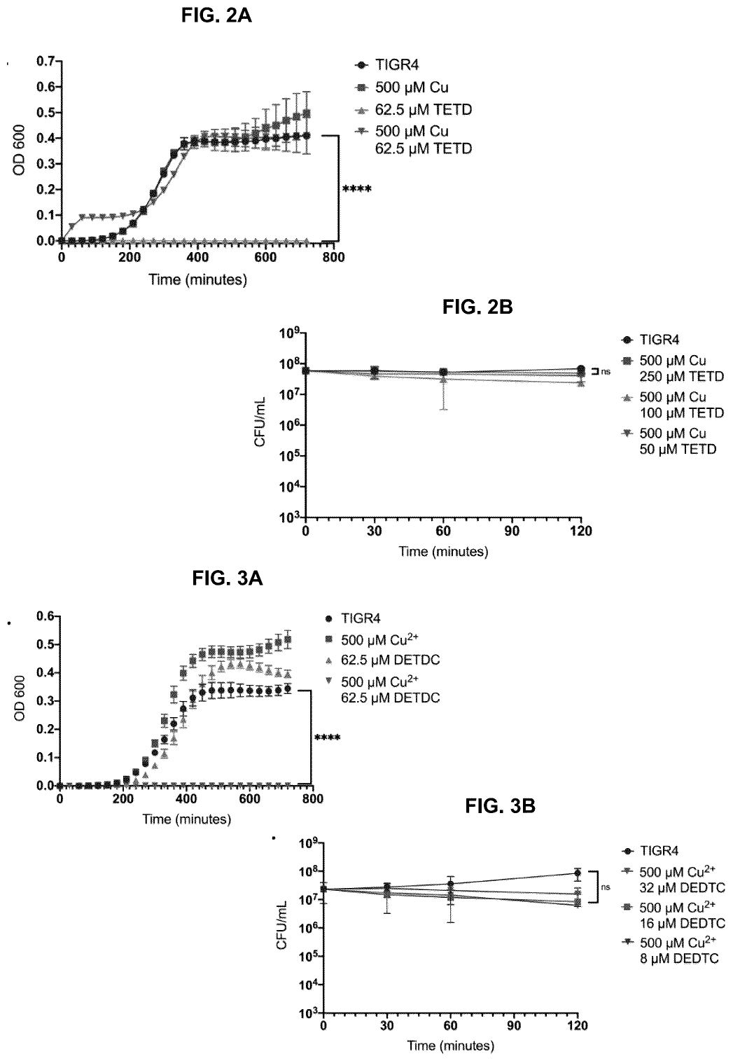

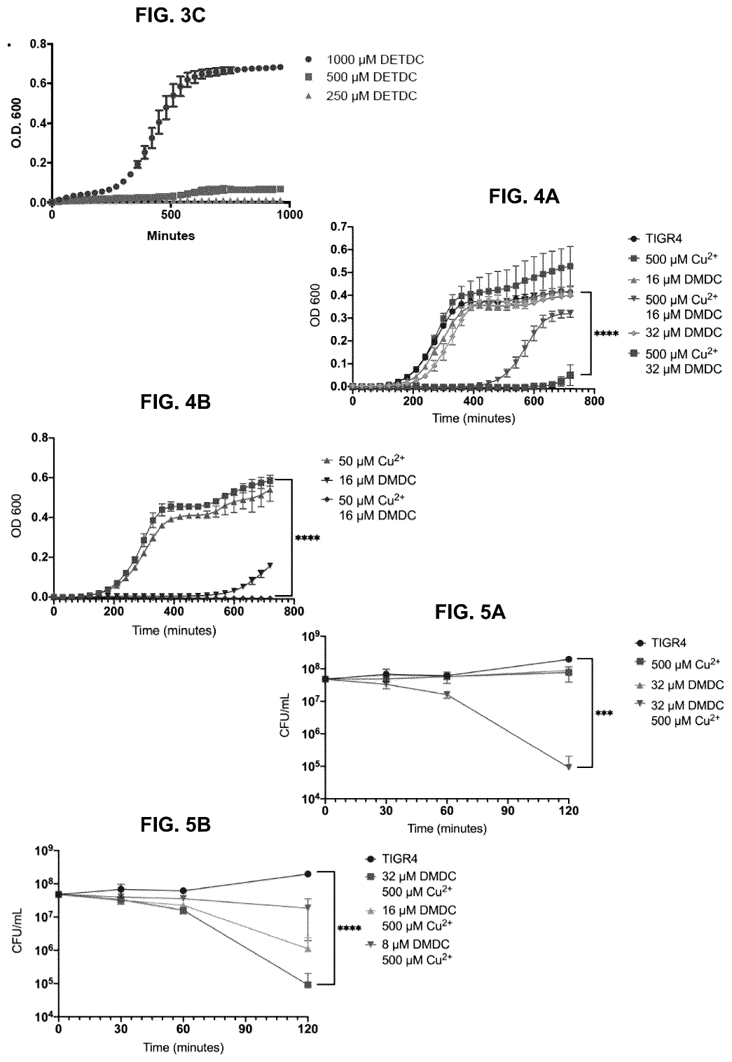

Disulfiram (Antabuse, tetraethylthiuram disulfide, TETD) has previously been seen to have copper dependent toxicity (CDT) against M. tuberculosis. TETD was tested to see if it had any CDT against the pneumococcus. While TETD alone prevented growth at multiple concentrations, adding copper returned growth to wild type levels (FIG. 2A). Further, TETD alone or combined with copper did not show bactericidal activity (FIG. 2B). TETD can be reduced to N,N-diethyldithiocarbamate (DETDC) within a matter of minutes inside the host. We found that while DETDC had CDT for concentrations under 250 μM, much higher concentrations were ineffective for reasons unknown (FIGS. 3A and 3B). Even so, there was no bactericidal activity measured in the killing curve, thus eliminating this compound as a viable CDT candidate moving forward (FIG. 3C).

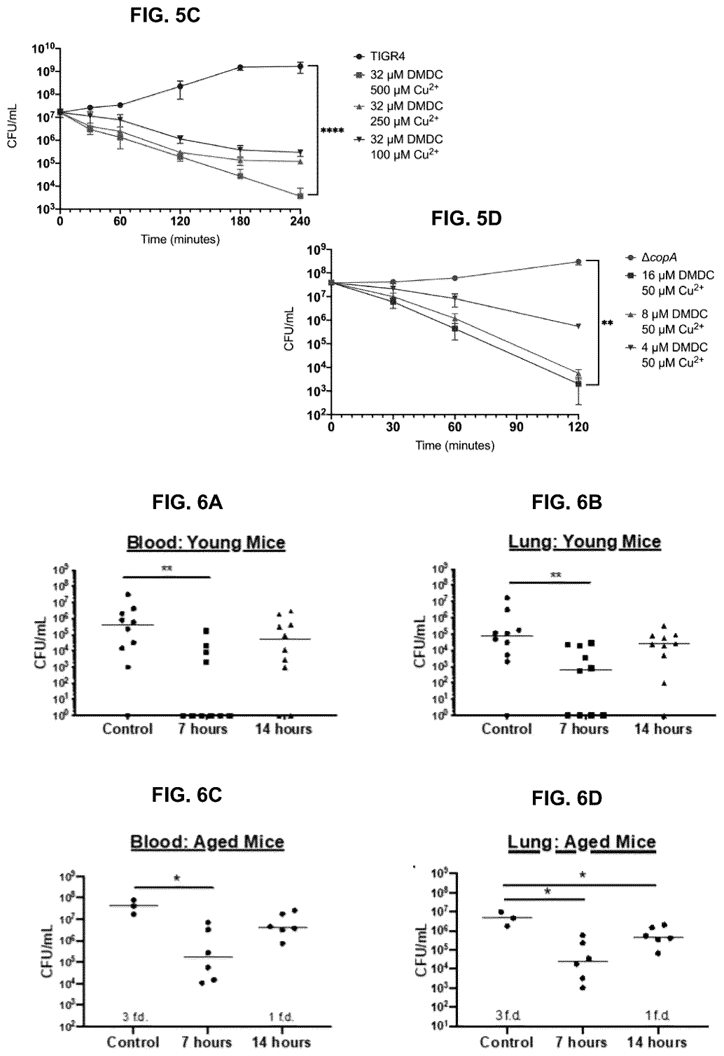

N,N-dimethyldithiocarbamate (DMDC) is a compound related to DETDC with methyl groups replacing ethyl groups. DMDC was examined for its ability to cause CDT in vitro, to determine if substitutions at this position would change the effects. In growth curves, CDT was observed for DMDC in a concentration-dependent manner with both TIGR4 and the ΔcopA mutant (FIGS. 4A and 4B). In the killing curve assay, bactericidal activity was observed in a DMDC-, copper-, and time-dependent manner with TIGR4 (FIGS. 5A, 5B, and 5C). Increased CDT was also observed in the killing curve with the ΔcopA mutant relative to TIGR4 (FIG. 5D).

A number of compounds that had no effect against the wild-type pneumococcus such as pyrithoxin dihydrochloride, no effect on wild type, but an effect on the ΔcopA mutant such as 1,10-phenalthroline monohydrate, as well as copper mediated protection against the compound and the protective synergistic switch. As none of these compounds displayed definitive CDT in the wild-type strain, only DMDC was studied further.

While the CDT efficacy of a compound against S. pneumoniae in a complex medium in vitro is promising, determining if DMDC works within an animal model is an important step to developing it for therapeutic use. The concentration of copper inside the infected lung is roughly 10 μM and 40 μM in infected blood. Given these concentrations and the nutrient-limited environment inside the host, DMDC was given at different concentrations and time points to test if bacterial burden could be reduced. The TIGR4 strain of S. pneumoniae is invasive and readily enters the bloodstream during lung infection. Therefore, both lung and blood titers were tested after two days post infection. Giving a lethal dose with 100% mortality to the mice intranasally, a significant decrease in bacterial titers after 48 hours was observed in mice that were given 25 μL of 10 mM DMDC intranasally (approximately 1.6 mg/kg) 7-hours post infection in the blood and lungs of 8-week-old mice (FIGS. 6A and 6B). The median titers of the 5 mM DMDC concentration and the 10 mM amount given at 14-hours post infection were lower, but the data was not significant (data not shown, FIGS. 6A and 6B).

As the pneumococcus is also particularly detrimental to elderly human populations, the ability of DMDC to reduce bacterial burden in 18-month-old geriatric mice was also tested. Here, significantly reduced titers in both blood and lung in the DMDC 7-hour post-infection groups were observed, and significantly reduced titers in the lung 14-hours post infection were observed as well (FIGS. 6C and 6D). Taken together, these data indicated DMDC is a viable candidate for treating pneumococcal lung infections.

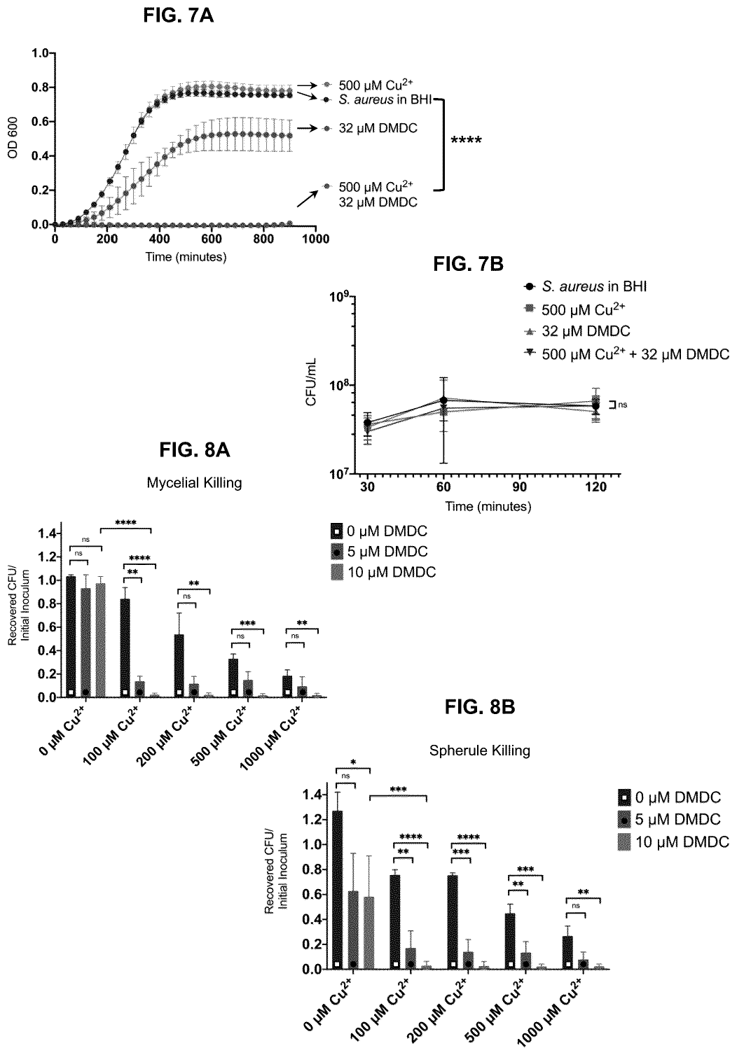

Next, how broad spectrum DMDC could be to other pathogenic organisms was determined. First, DMDC against S. aureus was tested. While S. aureus is a well-known cause of skin infections, toxic shock syndrome, and bacteremia, it can also cause pneumonia. 8HQ has been shown to have CDT against S. aureus. In a growth curve, while DMDC had inherent toxicity, the addition of copper caused completely ablated bacterial growth of S. aureus (FIG. 7A). However, upon performing the killing curve for 2 hours, no CDT was seen as compared to the control population, copper alone, or DMDC alone (FIG. 7B). Taken together, while DMDC is effective at preventing growth at the lag phase, however, with higher concentrations of bacteria in a different phase of growth, it was bacteriostatic at best, and had no effect at worst.

The next pathogen DMDC was tested against was the fungus C. posadasii. The life cycle of Coccidioides species is to transition from mycelia in the environment which generates arthroconidia and if inhaled, grow as spherules in the lungs. Endospores develop within spherules, and, with spherule rupture, each can propagate into a new spherule to perpetuate and expand the infection. While Coccidioides isn't spread from person-to-person, and can be suppressed by the host immune system, severe cases require antifungals and even so, is sometimes not enough to clear the potentially lifelong infection. DMDC was tested for CDT against C. posadasii in the mycelial and spherule stages. After varying concentrations of DMDC and copper (similar to bacterial killing curves) and observing a reduction in viable C. posadasii organisms, it was concluded that DMDC had CDT on both mycelial and spherule life stages in a copper and DMDC dependent manner (FIGS. 8A-8B). Taken together, based on this in vitro data, DMDC is a viable option for future therapeutic studies for C. posadasii and the other species of the genus, Coccidioides immitis. After demonstrating both antibacterial and antifungal effects of DMDC and through additional collaboration, DMDC was tested against an animal parasite.

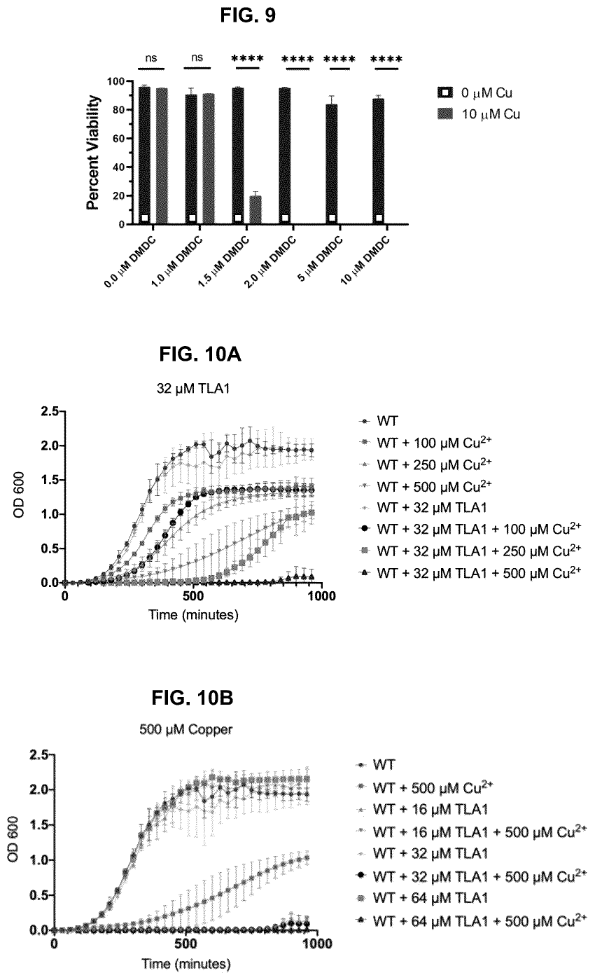

Schistosoma life cycles require both a molluscan intermediate host and a definite mammalian host. After adhering to host skin, their larvae called cercariae bore through the skin of mammals using proteases. The adult worms pair and mate, producing hundreds of eggs daily. Schistosomiasis, the host's immune response to these eggs, can lead to hepatosplenomegaly, pulmonary hypertension, urethral and bladder fibrosis, bladder and colorectal cancer, and death. The primary treatment for schistosome infection has been praziquantel, however, its efficacy in single dosage and noncompliance as a result of its taste and gastrointestinal side effects has created challenges in treatment. To test the CDT against S. mansoni, newly transformed and lung stage schistosomula were used; two developmental stages where praziquantel is not effective. DMDC was found to have CDT against S. mansoni in a compound dependent manner in both stages (FIG. 9). There was no reduction in viability at 10 μM DMDC without copper, however at concentrations as low as 2 μM DMDC with 10 μM copper, there was no viability (FIG. 9). Further, DETDC was also efficacious as an anti-helminthic of S. mansoni newly transformed and lung stage schistosomula at the same concentrations, but similarly to the results in the pneumococcus, not effective at higher concentrations. Taken together, CDT is a feasible therapeutic for a variety of pathogenic organisms.

The present invention demonstrated that N,N-diethyldithiocarbamate (DETDC) had potent CDT at mid-to-low micromolar concentrations (<100 μM) with low copper concentrations, but no effect with copper at high concentrations. The related compound N,N-dimethyldithiocarbamate (DMDC) displayed CDT at even lower tested concentrations of compound than DETDC against the pneumococcus. Further in vivo testing of DMDC against the pneumococcus using a murine model of pneumonia showed considerable efficacy against bacterial load. Lastly, in vitro efficacy of CDT with DMDC on other opportunistic pathogens was observed: Staphylococcus aureus, a gram-positive commensal known to harbor extreme antibacterial resistance; Coccidioides spp., endemic fungi to the southwestern United States known to cause San Joaquin Valley fever (often referred to as Valley fever); and Schistosoma mansoni, the parasitic flatworm which infects over 200 million people worldwide. Together, these data show that DMDC enhances Cu toxicity in human pathogens across three Kingdoms of life (bacterial, fungal, and animal).

EXAMPLE 2: The following is a non-limiting example of the present invention. It is to be understood that said example is not intended to limit the present invention in any way. Equivalents or substitutes are within the scope of the present invention.

Bacterial culture: M17 media (M17) (BD Difco, USA) was prepared according to manufacturer's instructions. Briefly 37.25 g of powder was suspended in 950 mL of Milli-Q grade water (≥18.0 MΩ cm-1) and autoclaved at 121° C. for 15 minutes before cooling to 50° C. and adding 50 mL of a sterile 10% lactose solution. Gibco Roswell Park Memorial Institute (RPMI) 1640 media containing L-glutamine and 4 g/L NaHCO₃ was purchased from the University of Arizona BIO5 Institute Media Facility. Prior to growth in RPMI, cold RPMI was supplemented with 0.1 mg/mL catalase, 30 mM glucose, 1× trace metals, and 1× “supplements” which include but are not limited to Uracil, Adenine, Glyce, Choline chloride, Sodium Carbonate, or a combination thereof. Tryptic Soy Agar (TSA) (Hardy Diagnostics, USA) was dissolved in Milli-Q water and autoclaved. After cooling the autoclaved TSA, 5% defibrillated sheep's blood (HemoStat Laboratories) of final volume and 20 μg/mL neomycin were added to the solution. These plates (blood agar plates—BAP), were used for routine culture on solid media and for “killing curve” serial dilution CFU counting. Bacteria from freshly-streaked plates were placed into M17 and grown at 37° C. in 5% CO₂, to an optical density (OD or OD600) of 0.125 for growth curve assays and to an OD of ˜0.300 for killing curve assays. To prepare working stocks of viable S. pneumoniae, growing cultures are resuspended in fresh media+20% v/v glycerol and stored at −80° C. Aliquot viability and CFU (colony-forming unit) counts were determined as discussed below before use in experiments. Glycerol stock aliquots were diluted 1:5 into M17 or RPMI with indicated copper and compound concentrations for assays.

Growth Curves: Copper stock solutions at 100 mM were prepared from CuSO₄ pentahydrate (VWR Life Sciences, USA) in Milli-Q water. Stock solutions of 100 mM Zn²⁺ were prepared from ZnSO₄ heptahydrate (VWR Life Sciences, USA). Stock solutions of 100 mM DMDC were prepared from Sodium Dimethyldithiocarbamate Dihydrate (Tokyo Chemical Industry, Japan) in Milli-Q water. Sterile, individually-wrapped, clear 96 well polystyrene plates (Greiner Bio-One, USA) were arranged to test a range of concentrations combinations of Cu²⁺ Zn²⁺, and DMDC. Frozen aliquots of S. pneumoniae were thawed and diluted five-fold into fresh M17 before adding 20 μL per well into a total well volume of 200 μL (1:50 total dilution). Assay plates were loaded into a Biotek Cytation5 (Biotek, Vermont, USA) pre-equilibrated to 37° C. and 4% CO₂. Gas control settings were modified for an elevation of 720 m according to manufacturer's directions. The protocol-maintained temperature and CO₂, while measuring OD absorbance at 600 nm every 30 minutes for 16-20 hours.

Killing Curves: Aliquots of S. pneumoniae were thawed and diluted ten-fold into assay conditions prepared in M17 or RPMI, respectively. Assay conditions included various concentrations of CuSO₄, DMDC, hydrogen peroxide (Sigma-Aldrich) and DPTA NONOate (Cayman Chemical Company, USA). After exposure to the indicated conditions, bacteria were incubated at 37° C. in 5% CO₂ for the indicated time, samples were serially diluted, plated on BAP, incubated overnight at 37° C. in 5% CO₂, and counted to determine viable CFU unless variations were specified in the specific figures. Colonies on each plate were counted and multiplied by the appropriate dilution factor based on which dilution it was to determine CFU/mL.

For plates in which no colonies were visualized at all, this was deemed to be below the limit of detection (LoD) and is noted with a data point below the LoD line.

Graphite Furnace Atomic Absorption Spectroscopy: Experiments were performed in triplicate. TIGR4 S. pneumoniae were initially cultured on M17+5 mM lactose and frozen at −80° C. in 20% glycerol. These glycerol stocks were used as the seed stock to inoculate 40 mL of M17+5 mM lactose. The bacterial culture was incubated at 37° C. under 5% CO₂ until an OD of ˜0.300 was reached. The culture was split into the indicated treatment and control. Incubation of treatments was performed at 37° C. and 5% CO₂ for 30 minutes. Samples were quenched in −3° C. water bath to slow down metabolism, followed by 2 washes of cold TBS (tris 50 mM, NaCl 150 mM, EDTA 50 mM at pH 7.6), and centrifugation 7800× g for 7 minutes at 4° C. Cold decanted samples were stored in −20° C. overnight before resuspension in 5% HNO₃. Bacterial plate counts were performed in TSA+5% Sheep's Blood through serial dilutions, as described above. Samples were analyzed for copper content using a Thermo iCE 3400 atomic absorption spectrometer with a 324.8 nM wavelength. Standards were made using TraceCERT® copper standard for AAS by Sigma-Aldrich and copper content of the washed samples was calculated based on average absorbance of at least six independent measurements with four resampling each. Baseline measurements of media alone were conducted to ensure instrument accuracy, finding copper levels to be consistent with that of the untreated control. Statistical significance was determined through unequal variances t-test with p values as follows: *p<0.05, **p<0.01, ***p<0.001, and ****p<0.0001.

Capsule Blot: Briefly, bacteria from freshly-streaked BAP were grown in M17 media to OD ˜0.400 prior to separating into 1 mL cultures and exposing to indicated conditions for 30 minutes. Equal CFU/mL were obtained for each condition. Following exposure, bacteria were pelleted by centrifuging at 3500× g for 10 minutes and resuspending the pellet in 1 mL of SMH buffer (0.5 M sucrose, 0.02 M MgCl2, and 0.02 M HEPES). Next, bacterial pellets were centrifuged at 14,000×g, treated with 100 μL of 10 mg/mL lysozyme (Gold Biotechnology, USA) and 20 μL of Proteinase K (Gold Biotechnology, USA) at room temperature for 10 minutes. Pellets were then exposed to 13 μL 10×SDS buffer, boiled for 10 minutes at 95° C., and 20 μL of each sample loaded onto a 0.8% agarose gel. Samples were transferred onto a mixed nitrocellulose ester membrane via 20×SSC capillary transfer overnight. Membranes were cross-linked at 150,000 mJ using a Stratagene UV Crosslinker, blocked for 1 hour in PBST with milk, probed with 1:1000 anti-capsular antiserum (SSI Diagnostica, serotype 4 cat 16747), washed with 1×PBST for 5 minutes 3 times, probed with 1:30,000 secondary antibody (horseradish peroxidase-conjugated), washed again in 1×PBST for 5 minutes 3 times, and imaged on an imager following addition of ECL (Cytiva, USA) as specified by the manufacturer.

Macrophage Killing Assays: J774A.1 macrophages (ATCC, USA) were maintained in a 37° C., 5% CO2 incubator with Dulbecco's Modified Eagle's Medium (DMEM; Sigma-Aldrich, USA) containing fetal bovine serum (FBS [10% vol/vol]; Sigma-Aldrich, USA), glutamine (2 mM; Sigma-Aldrich, USA), penicillin (50 units/ml; Sigma-Aldrich, USA), streptomycin (50 mg/ml; Sigma-Aldrich, USA), and NaHCO₃ (0.015%). Cells were grown to 90% confluence in 12-well tissue culture plates (Greiner CELLSTAR®, Greiner Bio-One, USA) in 1 mL/well of growth medium. On the morning of the experiment, macrophages were washed twice with 1 mL PBS, and resuspended in 1 mL of “Serum-Free DMEM” growth medium without antibiotics, glutamine, NaHCO₃ or FBS but supplemented with 5 ng/mL IFN-γ (Bio Basic, USA), 400 ng/mL LPS (EMD Millipore, MilliporeSigma, USA) for “priming of macrophages” experiment. For experiments involving treatment with apocynin (Santa Cruz Biotechnology, USA) or L-canavanine (Sigma-Aldrich, USA), “Serum-Free DMEM” was supplemented with 100 μM of each inhibitor as indicated. For “post hoc killing efficiency” experiment, macrophages were resuspended in 1 mL of Serum-Free DMEM containing 32 μM DMDC, 5 ng/mL IFN-γ, and 400 ng/mL LPS.

Macrophages were incubated at 37° C., 5% CO₂ for 12 hours. Glycerol stocks of TIGR4 S. pneumoniae kept at OD 0.3 are removed from −80° C. storage, diluted into four 15 mL conical tubes of 5 mL total M17+Lactose containing no additives (“Untreated”), 32 μM DMDC, 250 μM CuSO₄, and 250 μM CuSO₄+32 μM DMDC respectively for Pre-treatment of Bacteria experiment. Prior to incubation of bacteria in 37° C., 5% CO₂, an inoculum plate is made by serial diluting 100 μL from the no additives conical. After 15 minutes of incubation, bacteria are centrifuged at 4500×g for 10 minutes and resuspended in DMEM without antibiotics, glutamine, NaHCO₃ or FBS. Macrophages are removed from incubation, media is removed, washed with 1 mL PBS twice and then infected with 100 μL of S. pneumoniae solutions for both experiment types, corresponding to a multiplicity of infection (MOI) of 10 bacteria per macrophage. The 12-well tissue culture plates were centrifuged at 200×g for 2 minutes to facilitate co-culturing.

Wells were then washed twice with PBS at the given time-points; each wash was followed by a 5-min incubation in a 37° C., 5% CO₂ incubator in DMEM containing gentamicin (50 μg/ml). Macrophages were lysed in 0.02% SDS in ddH₂O and serially diluted to determine the counts of viable intracellular bacteria. Data were normalized to the level of killing observed for the untreated TIGR4 bacteria for each assay.

Animal experiments: All mouse studies were conducted with prior approval and under the guideline of the Institutional Animal Care and Use Committee at the University of Arizona, IACUC protocol number 18-410, R35 GM128653. All mice were maintained in a biosafety level 2 (BSL2) facility and monitored daily for signs of moribund. Eight-week old female BALB/cJ mice (Jackson Laboratory, USA) were anesthetized with 3% isoflurane and intranasally given either: 1) 25 μL of Tris-Buffered Saline (TBS—50 mM Tris, 150 mM NaCl, pH 7.4), 2) 0.8 mg/kg DMDC in 25 μL TBS, 3) an inoculum of 1×10⁷ CFU viable S. pneumoniae in 25 μL of TBS, or 4) 1×10⁷ CFU viable S. pneumoniae in 25 μL TBS and subsequent 0.8 mg/kg DMDC in 25 μL TBS. Control TBS and bacterial infections were carried out 8 hours prior to mice given DMDC. For Group 4, mice were intranasally infected before being treated with DMDC approximately 8 hours later. Mice were sacrificed by CO₂ asphyxiation and immediately dissected for lung and blood collection 48 hours post infection and treatment. Lung tissue was collected into 1.5 mL tubes, containing 500 μL Phosphate Buffered Saline (DPBS, Gibco, USA). Single-cell suspensions were prepared from lung tissue as described below.

Preparation of single-cell suspension from lung: Briefly, lungs were perfused with PBS and finely minced before being placed into digestion buffer containing 1 mg/mL Collagenase D (Millipore Sigma, Darmstadt, Germany) and 0.15 mg/mL DNase I (Sigma-Aldrich, USA) in DMEM (HyClone, Sigma-Aldrich, USA). Lungs were digested for 20-25 minutes at 37° C. at 200 RPM then passed through a 40 μm cell strainer to prepare single-cell suspension.

Antibodies and Flow Cytometry: For surface staining, fluorophore-conjugated mAbs specific for CD11b (clone M1/70), CD45 (clone 30-F11), F4/80 (clone BM8), and Ly6G (clone 1A8) were obtained from BioLegend (USA) and fluorophore-conjugated mAb specific for CD11c (clone HL3) was obtained from BD Biosciences (USA). Cells were run on an LSRII (BD Biosciences, USA), and analyses were performed with FlowJo (TreeStar, BD Biosciences, USA) software.

Inductively Coupled Plasma Optical Emission Spectroscopy: Experiments were performed in triplicate. TIGR4 S. pneumoniae were initially cultured on M17+5 mM lactose and frozen at −80° C. in 20% glycerol. These glycerol stocks were used as the seed stock to inoculate 150 mL of M17+5 mM lactose. The bacterial culture was incubated at 37° C. under 5% CO₂ until an OD of ˜0.400 was reached. The culture was split into the indicated treatment and control. Incubation of treatments was performed at 37° C. and 5% CO₂ for 30 minutes. Samples were quenched in −3° C. water bath to slow down metabolism, followed by 2 washes of cold TBS (tris 50 mM, NaCl 150 mM, EDTA 50 mM at pH 7.6), and centrifugation 3500× g for 10 minutes at 4° C. Cold decanted samples were resuspended in 2% HNO₃. Bacterial plate counts were performed in TSA+5% Sheep's Blood through serial dilutions, as described above. Samples were analyzed for metal content using an iCAP PRO XDUO ICPOES with a wavelength 324.8 nm copper, 213.8 nm for zinc, 257.6 nm for manganese and 393.3 nm for calcium. Standards were made using iCap Series Multi-element test solution ICAP 6000 series Validator from Thermo Scientific (Thermo Scientific, USA) and metal content of the washed samples was calculated using the Qtegra software. Baseline measurements of media alone were conducted to ensure instrument accuracy, finding copper levels to be consistent with that of the untreated control.

Statistical Analysis: Statistical significance was analyzed using Student t test (two-tailed, unpaired), two-way ANOVA, or one-way ANOVA and Dunnett's multiple comparisons test (Prism 9.20; GraphPad Software, USA). The p values were as follows: *p<0.05, **p<0.01, ***p<0.001, and ****p<0.0001.

### Results

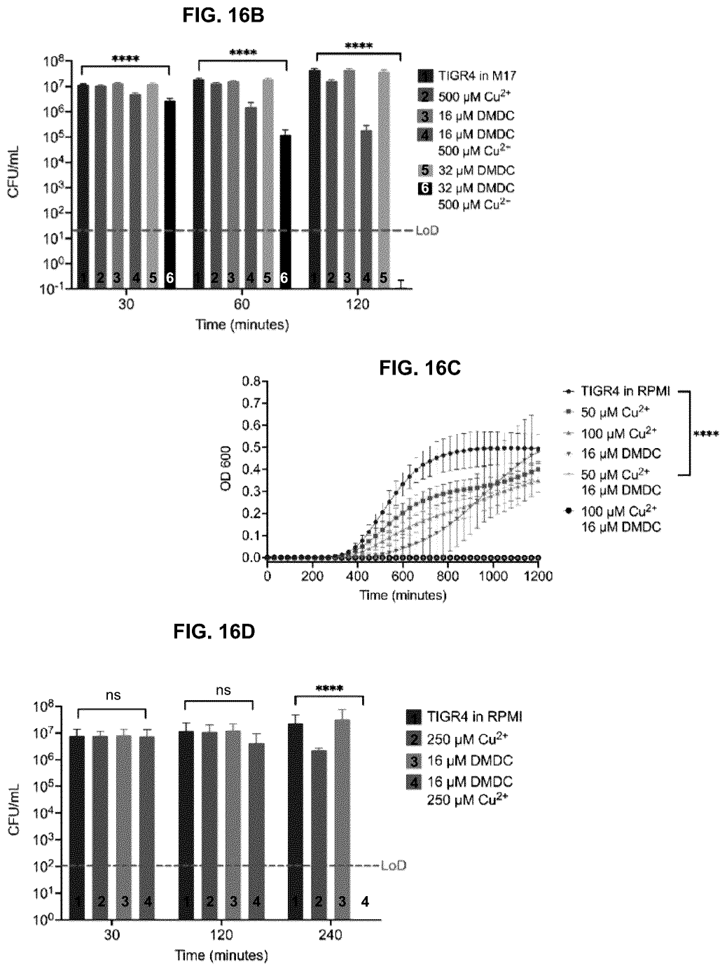

DMDC is a copper-dependent antibiotic in nutrient-rich (M17) and host-niche-mimicking media (RPMI): As previously discussed in Example 1, a targeted small molecule screen for compounds with ionophoric properties that can serve as a copper-dependent antibiotic against the pneumococcus was conducted. Additionally, the composition of growth media for S. pneumoniae was reviewed exposing heterogeneity in the characteristics of bacteria grown in nutrient-rich media (like Todd-Hewitt Broth [ThyB] and M17 media) in comparison to a more “minimal” host-niche-mimicking media (like RPMI). A major distinguishing factor between the two nutrient-rich media (ThyB and M17) is that M17 is prepared and sterilized without a carbon source; the manufacturer suggests 10% lactose solution or an alternative carbon source can be added after sterilization to provide greater control over media composition for an investigator. Growth curves and killing curves to further examine this dichotomy were performed utilizing nutrient-rich M17 media and host-niche-mimicking RPMI, as RPMI is traditionally used for cell culture of lung epithelial cells and leukocytes. In vitro growth curve assessment of TIGR4 S. pneumoniae in M17 media supplemented with copper±DMDC demonstrated a significant growth defect observed with the combination of 500 μM Cu²⁺+32 μM DMDC (FIG. 16A), which is equivalent to the combination required for toxicity seen previously in ThyB. Incubation with the combination condition obtained a significant decrease in CFU/mL to a level below our level of detection at t=120-minutes, indicating bactericidal activity for the 500 μM Cu²⁺+32 μM DMDC combination (FIG. 16B). DMDC was identified as a copper-dependent antibiotic shown in vivo efficacy in a murine pneumonia model (see Example 1). Since the compound has efficacy in vivo, how this compound works in a more nutrient-restricted and host-niche-mimicking media, such as RPMI 1640, was explored further. An in vitro growth curve assessment of TIGR4 S. pneumoniae in RPMI media supplemented with copper±DMDC demonstrated a significant growth defect observed with the combination of 50 μM Cu²⁺+16 μM DMDC (FIG. 16C). TIGR4 S. pneumoniae in RPMI media supplemented with copper±DMDC in a killing curve demonstrated a bactericidal combination of 250 μM Cu²⁺+16 μM DMDC at the t=180-minute and t=240-minute timepoints (FIG. 16D). For combinations of copper+DMDC utilizing less than 250 μM Cu²⁺, no killing effect—bactericidal or bacteriostatic was observed (data not shown). It is interesting to note that the time required for killing is longer in RPMI (3-4 hours) than in M17 (1-2 hours) (FIGS. 16B and 16D). These data demonstrate that DMDC is a copper-dependent bactericidal antimicrobial in various growth media ranging from host-niche-mimicking to nutrient-rich media.

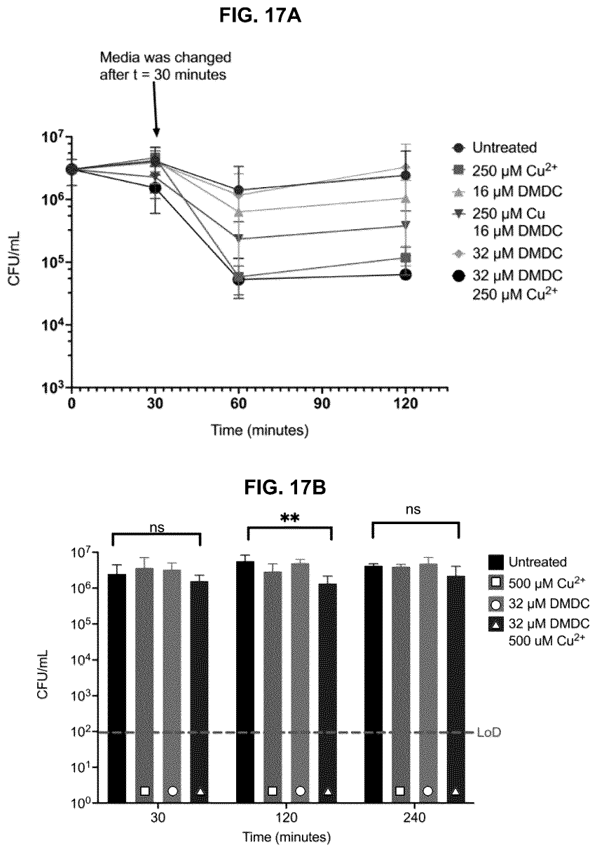

DMDC's bactericidal activity requires constant exposure and is temperature dependent: Next, how DMDC interacts with the bacterium was examined. To test this, TIGR4 S. pneumoniae were incubated in supplemented M17 media for 30 minutes before pelleting the bacteria and resuspending in fresh media lacking supplementation. While the pneumococcus continues to show decreased CFU/mL at the 60-minute timepoint in the combinatory DMDC+copper conditions the bacterial counts remained static (FIG. 17A) as opposed to seeing continued killing the 120-minute time point of FIG. 16B.

As temperature is a contributing factor to active transport with lower temperatures inhibiting active transport, this principle was extended to perform a killing curve in an ice-bath (4° C.) to prevent DMDC from entering the cell. Without active transport, the combination of copper+DMDC would not be able to work as efficiently as a bactericidal antibiotic. At 4° C., 500 μM Cu²⁺+32 μM DMDC no longer killed the pneumococcus as it did at 37° C. (FIG. 17B, and FIG. 16B). There is a statistically significant difference in bacterial CFU counts between untreated and combination conditions at t=120-minutes, but this significance is not present at t=240-minutes. Overall, these data show that DMDC's copper-dependent toxicity requires constant exposure to a bacterium and that this toxicity is temperature-dependent, indicating that this compound is likely actively transported into the bacterium.

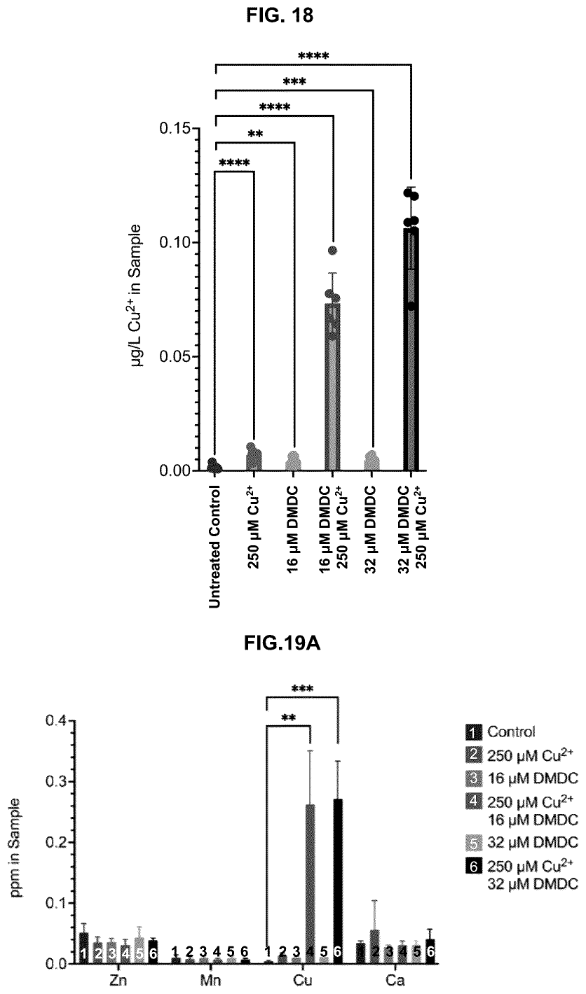

Exposure to copper+DMDC causes an increase in intracellular copper: To determine DMDC's effect on copper intoxication on the pneumococcus, the concentration of copper was quantified within the bacterium after treatment with 250 μM Cu²⁺±16 or 32 μM DMDC. Compared to the untreated control, there is a statistically significant increase in copper content within the bacterium of 250 μM Cu²⁺+16 μM DMDC-treated bacteria and within the bacterium of 250 μM Cu²⁺+32 μM DMDC-treated bacteria (FIG. 18). Compared to the copper treated samples, the 250 μM Cu²⁺+16 μM DMDC-treated bacteria and the bacterium of 250 μM Cu²⁺+32 μM DMDC have a 7- and 10-fold increase of intracellular copper, respectively.

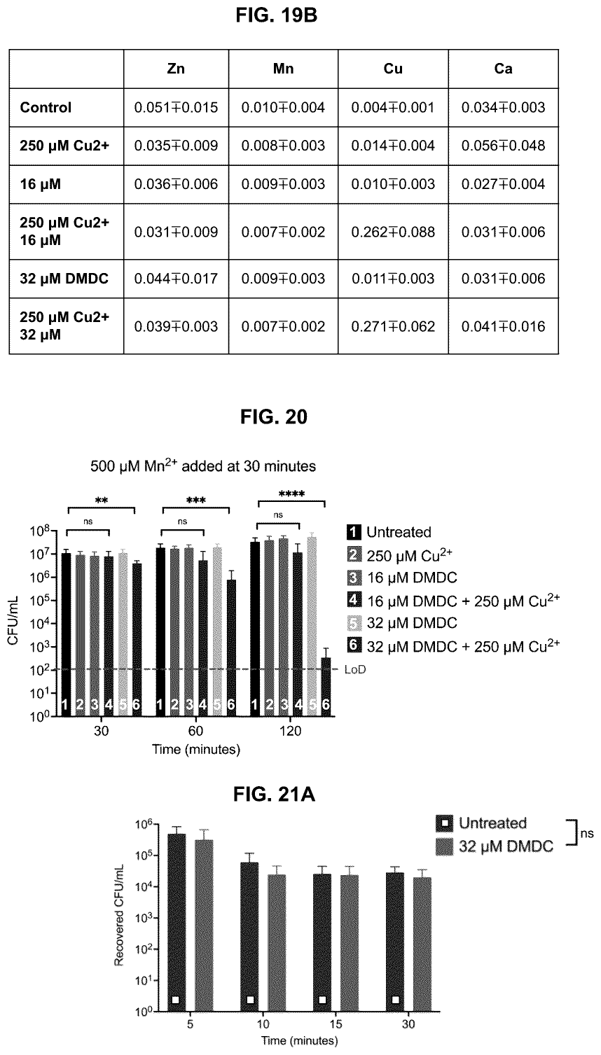

To further investigate the concentrations of intra-bacterial nutrient metals, Inductively Coupled Plasma-Optical Emission Spectroscopy (ICP-OES) was utilized to quantify concentrations of zinc, manganese, copper, and calcium. Compared to the untreated control, there is a statistically significant increase in copper content within the bacterium of 250 μM Cu²++16 μM DMDC-treated bacteria and within the bacterium of 250 μM Cu²⁺+32 μM DMDC-treated bacteria (FIG. 19A). Interestingly, the ICP-OES method determined that 250 μM Cu²⁺+16 μM DMDC-treated bacteria and the bacterium of 250 μM Cu²⁺+32 μM DMDC have a 65- and 67-fold increase of intracellular copper, respectively, compared to the untreated control. Additionally, there was no statically significant difference in intra-bacterial concentration of zinc, manganese, or calcium for any condition comparison. FIG. 19B shows a table of the mean±standard deviation for the experiments. Given these findings, these data suggest that treatment with DMDC and copper leads to increased intracellular copper concentration and thus, an increase in copper stress experienced by the bacterium.

DMDC's copper-dependent toxicity can be rescued by manganese supplementation: DMDC exacerbates mismetallation as a mechanism of action via the addition of manganese rescuing DMDC's copper-dependent toxicity in vitro (Example 1). To directly test if copper-dependent toxicity associated with DMDC can be rescued by manganese supplementation post-DMDC and copper addition, a killing curve was performed in which manganese was added at t=30-minutes (FIGS. 19A and 19B). While the addition of 500 μM Mn²⁺ at t=30-minutes rescued the 250 μM Cu²⁺+16 μM DMDC condition, it did not rescue the 250 μM Cu²⁺+32 μM DMDC condition (FIGS. 16A, 16B, 16C, and 16D). Thus, the ability to rescue copper-dependent toxicity mediated by mismetallation exists as expected but with limitations. Nevertheless, these data provide further evidence for DMDC exacerbating copper-dependent mismetallation toxicity within the bacterium.

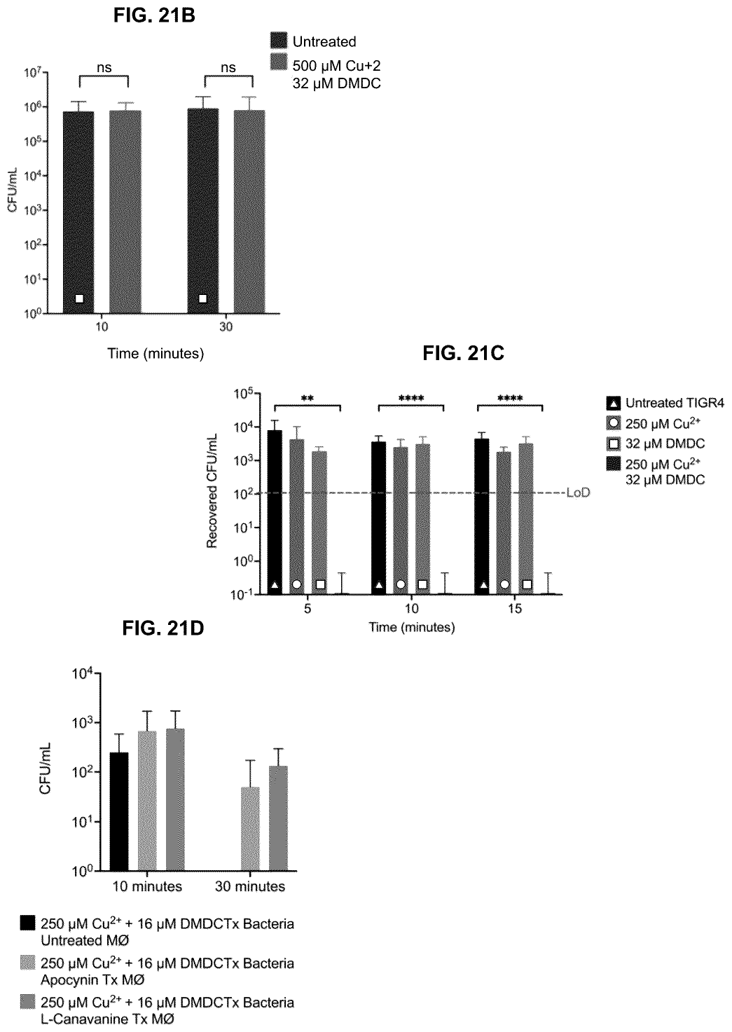

DMDC and copper-treated TIGR4 S. pneumoniae are killed at a faster rate by J774A.1 murine macrophages than untreated bacteria: Since the copper-dependent toxicity of DMDC is enhanced by incubation in a host-niche-mimicking media (FIGS. 16C and 16D), and macrophage-mediated clearance is a key mechanism of innate immune clearance of pathogenic S. pneumoniae, next it was determined if in vitro incubation with murine macrophages leads to enhanced macrophage bactericidal activity. First, if DMDC is cytotoxic to macrophages was examined. J774A.1 macrophages were exposed to the highest used DMDC concentration and found not to be toxic via Trypan blue cytotoxicity assay (FIG. 20). From there, we wanted to test if DMDC works by priming macrophages for improved killing. We treated macrophages with 32 μM DMDC while activating the macrophages (treating with LPS and IFN-γ) 6 hours before co-culturing with S. pneumoniae. No statistical difference between the recovered CFU/mL of bacteria from co-culturing with the treated macrophages and the untreated control (FIG. 21A). It was further tested if adding increased copper was necessary along with DMDC exposure to generate an improved killing rate. Incubation of macrophages with a combination of 500 μM Cu²++32 μM prior to co-culture with S. pneumoniae did not show a significant difference in killing rate compared to untreated control macrophages (FIG. 21B).

Next, if DMDC can increase the efficiency of post hoc killing of antibiotic-treated bacteria by macrophages was tested. In M17, all bacteria were still viable at 15 minutes at the highest dosages of DMDC+copper (data not shown). Thus, the bacteria treated with 250 μM Cu²⁺+32 μM DMDC were incubated for 15 minutes prior to co-culturing with macrophages for 5-, 10-, and 15-minute incubations to test macrophage killing ability. The bacteria recovered at each timepoint for the DMDC+copper treatment was below the limit of detection (FIG. 21C). As such, the recovered CFU counts show a statistically significant difference between the untreated and copper+DMDC-treated groups. Overall, these data indicate that DMDC aids in the macrophage killing rate post hoc of copper+DMDC-treated bacteria.

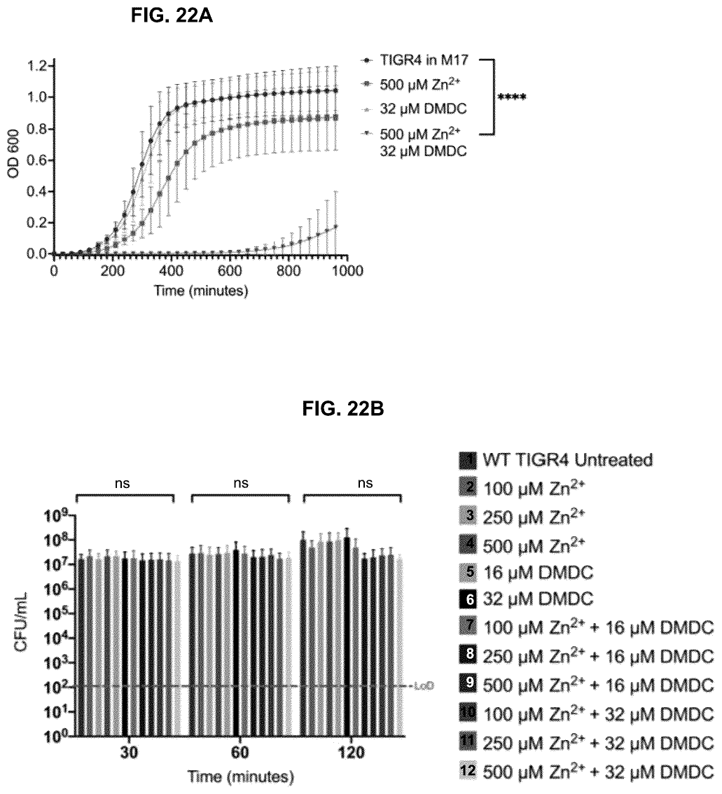

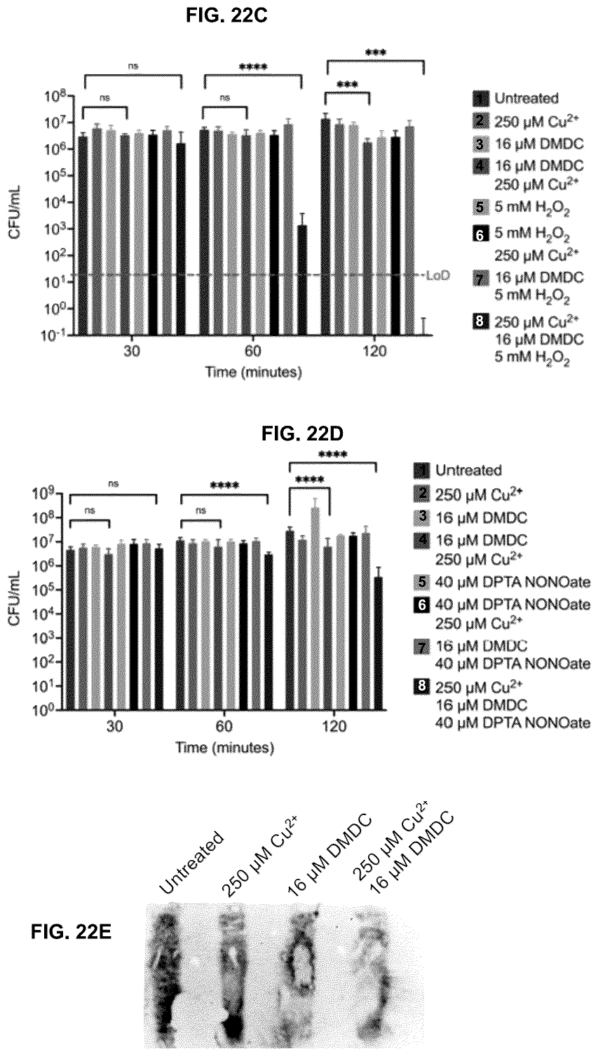

To test if the improvement in macrophage post hoc bacterial clearance is mediated by macrophage phagolysosomal nitric oxide and reactive oxygen species, inhibitors of these killing mechanisms were utilized as shown in FIG. 22D. Macrophages were incubated with 100 μM apocynin to inhibit oxidative killing. Alternatively, macrophages were incubated with 100 μM L-canavanine to inhibit nitric oxide. Treated and untreated macrophages were given bacteria treated with 250 μM Cu²⁺+16 μM DMDC (this was less than the levels used in FIG. 21C). There was no statistically significant improvement in recovered bacteria with the macrophage treatment, however there was a trend towards an improvement. Taken together, these mechanisms may partially contribute to the improved post hoc bacterial clearance.

In vitro incubation of DMDC in conditions replicating the macrophage phagolysosome display enhanced susceptibility to copper-dependent toxicity: To mediate killing of pathogens within the phagolysosome, macrophages utilize high concentrations of zinc, hydrogen peroxide, nitric oxide, and copper as well as having a low pH and proteases. To further understand why the pre-treatment of bacteria led to a rapid J774A.1 macrophage clearance of bacteria, growth curve and killing curve assays were performed in vitro to mimic the environment of the macrophage phagolysosome. A growth curve with TIGR4 S. pneumoniae in M17 media supplemented with indicated combinations of zinc and DMDC was performed and found that there is a growth defect for the 500 μM Zn²⁺+32 μM DMDC condition (FIG. 22A). A killing curve was also performed in M17 media with a titration of combinations of zinc±DMDC, showing that the 500 μM Zn²⁺+32 μM DMDC condition is bacteriostatic—there is no significant difference in CFU/mL between the untreated control and the combination treatment (FIG. 22B).

To test the contribution of hydrogen peroxide in the macrophage killing seen in FIG. 22B, a killing curve was performed in which M17 media was supplemented with combinations of copper, DMDC, and hydrogen peroxide (FIG. 22C). Utilizing a lower amount of copper (250 μM compared to 500 μM used in previous figures), lower amount of DMDC (16 μM compared to 32 μM utilized in previous figures), and 5 mM hydrogen peroxide to which S. pneumoniae TIGR4 displays control level of growth, the combination of 5 mM H₂O₂+250 μM Cu²⁺+16 μM DMDC displayed robust killing at t=60-minutes that extended into t=120-minutes (FIG. 22C).

To test the contribution of nitric oxide in the macrophage killing seen in FIG. 21B, a killing curve was performed by supplementing M17 media with combinations of copper, DMDC, and a nitric oxide-donating compound, DPTA NONOate. Macrophages produce around 40 μM nitric oxide when activated by IFN-γ and LPS. For this reason, the killing curves were performed with combinations of 40 μM DPTA NONOate in FIG. 22D. The combination of 40 μM DPTA NONOate+250 μM Cu²⁺+16 μM DMDC displayed statistically significant killing at t=60-minutes and t=120-minutes as compared to both control and DMDC+copper treated bacteria. Overall, these data show that high zinc concentration, hydrogen peroxide exposure, and nitric oxide exposure combine with copper-dependent toxicity of DMDC to explain the rapid killing of bacteria when co-cultured with macrophages in vitro.

Combination treatment with DMDC and Cu²⁺ leads to a decrease in the amount of extracellular capsule of S. pneumoniae: The extracellular polysaccharide capsule of S. pneumoniae is a well-characterized mechanism against innate immune cell phagocytosis and against macrophage-mediated killing. Hydrolysis of the capsule of Type 3 S. pneumoniae renders the bacteria more susceptible to phagocytosis by macrophages and complement-mediated killing by neutrophils. Following established protocols for blotting against the Type 4 capsule of the TIGR4 strain of S. pneumoniae, FIG. 22E shows that the combination treatment of DMDC+Cu²⁺ leads to a decrease in observed capsule in the pellet fraction of bacteria. There was no capsule observed in the supernatant (data not shown). These data provide evidence that the enhanced macrophage killing due to treatment with DMDC+Cu²⁺ seen in FIG. 21B is due to a decrease in bacterial capsule that potentially allows for increased macrophage opsonization.

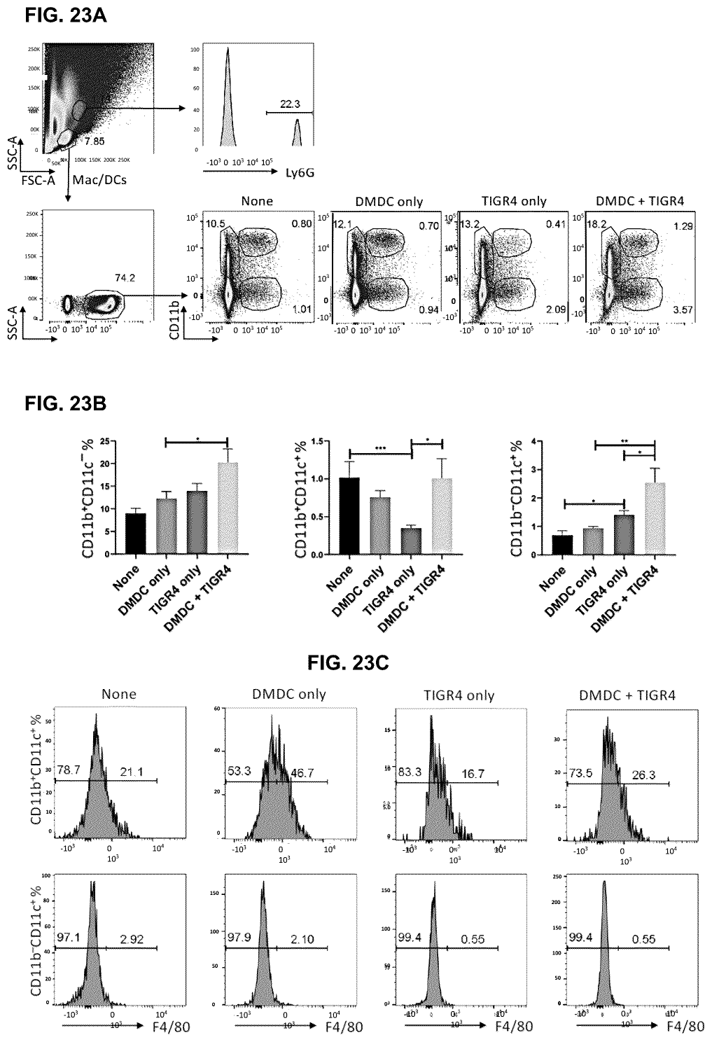

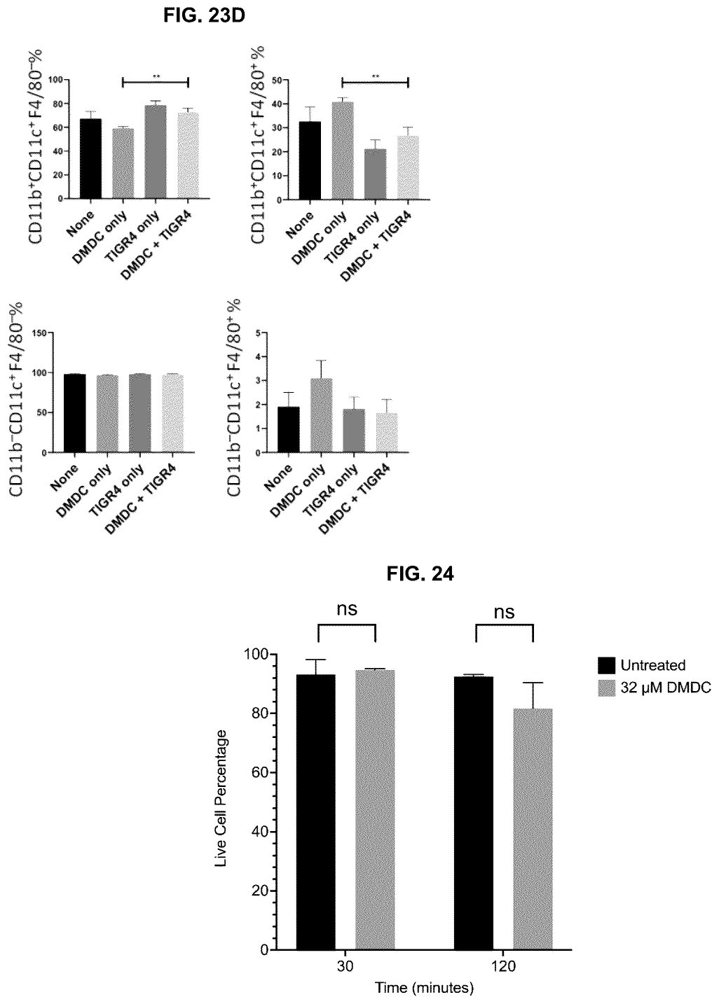

In vivo treatment of DMDC increases lung macrophages and dendritic cell populations upon S. pneumoniae infection: Treatment with DMDC alone significantly decreased bacterial burden in a murine pneumonia model of infection (Example). Here, the in vivo effect of DMDC on the macrophage and dendritic cell (DC) populations of mice treated with DMDC was examined. Cohorts of 8-week-old female BALB/cJ mice were treated with four conditions: 1) no treatment, 2) DMDC only, 3) TIGR4 only, and 4) DMDC+TIGR4. Lung single-cell suspensions were stained and gated on the indicated FSC/SSC area to avoid the cell population enriched with infiltrating L6G+neutrophils (FIG. 23A). The cells were further gated on CD45 for leukocytes and then CD11b versus CD11c, which can group cells into 3 subsets: 1) CD11b⁺CD11c⁻, 2) CD11b⁺CD11c⁺, and 3) CD11b⁻CD11c⁺ populations (FIG. 23A). Group 1 is enriched with monocytes and a few neutrophils, Group 2 is enriched with interstitial macrophages, and Group 3 contains both alveolar macrophages and dendritic cells. A significant increase in Group 2 CD11b⁻CD11c⁺ and Group 3 CD11b⁻CD11c⁺ populations in DMDC+TIGR4-treated lungs was seen compared to the TIGR4-only treated group (FIG. 23B). As seen in Group 2, DMDC treatment seemed to restore the interstitial macrophage population to wild-type levels as it was reduced in the TIGR4 alone condition. Group 3 was further gated into F4/80+ and F4/80− groups for alveolar macrophages and DCs respectively and found that, unlike Group 2 CD11b⁺CD11c⁺ cells, Group 3 CD11b⁻CD11c⁺ cells were mostly F4/80 negative and thus was enriched with dendritic cells (FIGS. 23C and 23D). Overall, these data show that DMDC treatment, either through interaction with the bacteria or the immune system, resulted in more immune cells to the lung environment, which corresponded with the reduction of bacterial burden in vivo.

Clinically, it is difficult to untangle if an antibiotic works to increase macrophage clearance of pathogens by working with macrophages directly or on bacteria to allow for macrophage post hoc clearance. The present invention first discovered that DMDC works on the pneumococcus in vitro independent of macrophages (FIGS. 16A, 16B, 16C, and 16D) before using co-culture experiments to answer this question for DMDC directly (FIGS. 21A, 21B, 21C, and 21D). DMDC treatment with copper sensitizes the pneumococcus to macrophage killing by increasing the internal copper concentration (FIG. 18 and FIGS. 19A and 19), increasing susceptibility to phagolysosomal weapons H₂O₂ and nitric oxide (FIGS. 22C and 22D), and decreasing the size of the antiphagocytic pneumococcal capsule (FIG. 22E). Additionally, in vivo administration of DMDC to mice upon pneumococcal infection increases the lung myeloid cell populations within the lung, corresponding with the reduction of bacterial burden. While changing the environment of the bacteria away from the DMDC+copper condition allows for the bacteria to eventually begin recovery (FIGS. 16A, 16B, 16C, and 16D), inadequate replenishing environmental nutrients under significant stress quickly led to a reduction in the capsule.

While bacteria struggle with mismetallation due to copper stress, they must also battle to eliminate the threat by exporting copper. In oxidizing environments, bacteria reducing copper for export comes at the cost of its reducing environment, which, if not replenished, will lead to dire consequences. While bacteria can import environmental carbohydrates, the nearest source for the pneumococcus is the capsule. The capsule is protection against future threats, such as the macrophage; however, the immediate threat of increasing the ability to export copper is the more pressing issue to survival. Thus, the pneumococcus cannibalizes its capsule for nutrients, making it far more susceptible to macrophage recognition (and therefore recruiting other immune cells), opsonization, and, ultimately, killing mechanisms.

EXAMPLE 3: The following is a non-limiting example of the present invention. It is to be understood that said example is not intended to limit the present invention in any way. Equivalents or substitutes are within the scope of the present invention.

### Examples and Synthesis of DMDC Analogs

### Example 3.1 Potassium morpholine-4-dithiocarbamate (TLA1, LRS01-043/046)

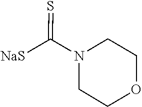

Potassium hydroxide (KOH) (56 mg, 1.00 mmol) was dissolved in ethanol (EtOH) (10 mL) and then cooled to 0° C. Morpholine (87 μL, 1.00 mmol) and carbon disulfide (CS2) (151 μL, 2.50 mmol) were added to the solution successively. The resulting mixture was stirred at room temperature for 2 hours, and then the solvent was reduced under vacuum. The crude was washed by cold diethyl ether (Et₂O) to give TLA1 (182 mg, 90%) as a white solid. 1H NMR (400 MHz, DMSO-d6) δ 4.34-4.22 (m, 4H), 3.51-3.39 (m, 4H). 13C NMR (100 MHz, DMSO-d6) δ 215.02, 66.58, 50.01.

### Example 3.2 Piperizine bis-dithiocarbamate (TLA2, LRS01-057, PBDC)

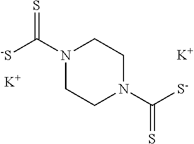

KOH (130 mg, 2.32 mmol) was dissolved in EtOH (10 mL) and then cooled to 0° C. Piperazine (91 μL, 1.16 mmol) and CS2 (350 μL, 5.80 mmol) were added to the solution successively. The resulting mixture was stirred at room temperature for 2 hours, and then the solvent was reduced under vacuum. The crude was washed by cold Et₂O to give TLA2 (210 mg, 57%) as a white solid. 1H NMR (400 MHz, DMSO-d6) δ 4.20 (s, 8H). 13C NMR (100 MHz, DMSO-d6) δ 214.14, 49.51.

### Example 3.3: Sodium 4-(p-tolyl)piperazine-1-carbodithioate (TLA3, LRS01-084)

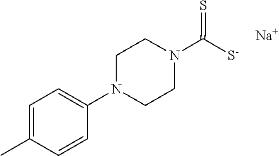 [SMILES: Cc1ccc(N2CCN(C(=S)[S-])CC2)cc1 ; [Na+]]

Sodium hydroxide (NaOH) (40 mg, 1.00 mmol) was dissolved in EtOH (6 mL) and then cooled to 0° C. 1-(p-tolyl)piperazine (176 mg, 1.00 mmol) and CS2 (151 μL, 2.50 mmol) were added to the solution successively. The resulting mixture was stirred at room temperature for 2 hours, and then the solvent was reduced under vacuum. The crude was washed by cold Et₂O to give TLA3 (230 mg, 83%) as a light yellow solid. 1H NMR (400 MHz, DMSO-d6) δ 6.98 (d, J=8.1 Hz, 2H), 6.80 (d, J=8.2 Hz, 2H), 4.41 (t, J=5.1 Hz, 4H), 2.96 (t, J=5.1 Hz, 4H), 2.16 (s, 3H). 13C NMR (100 MHz, DMSO-d6) δ 214.73, 149.41, 129.78, 127.97, 116.15, 49.25, 49.09, 20.49.

### Example 3.4: Sodium N-benzyl-N-methyldithiocarbamate (TLA4, LRS01-075, BMDC)

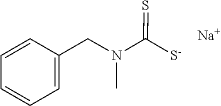

This compound was synthesized similar to the procedure of TLA3. White solid. Yield: 73%. 1H NMR (400 MHz, DMSO-d6) δ 7.28-7.19 (m, 4H), 7.19-7.12 (m, 1H), 5.43 (s, 2H), 3.24 (s, 3H). 13C NMR (100 MHz, DMSO-d6) δ 215.70, 139.58, 128.42, 127.66, 126.75, 57.85, 40.99.

### Example 3.5.1: Sodium N-allyl-N-methyldithiocarbamate (TLA5, LRS01-077, AMDC)

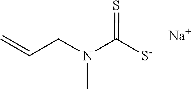

This compound was synthesized similar to the procedure of TLA3. White solid. Yield: 56%. 1H NMR (400 MHz, DMSO-d6) δ 5.83-5.70 (m, 1H), 5.08-5.02 (m, 1H), 5.02-4.97 (m, 1H), 4.73 (d, J=5.8 Hz, 2H), 3.25 (s, 3H). 13C NMR (101 MHz, DMSO-d6) δ 214.76, 135.06, 116.16, 57.57, 40.88.

### Example 3.5.2: Sodium methyl(2-methylallyl)carbamodithioate (TLA5-1, MW=189.328 g/mol)

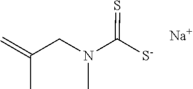

This compound is an analog of TLA5 and was synthesized using a similar procedure.

### Example 3.5.3: Sodium diallylcarbamodithioate (TLA5-2, MW=201.3318 g/mol)

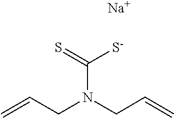

This compound is an analog of TLA5 and was synthesized using a similar procedure.

### Example 3.5.4: Sodium allyl(benzyl)carbamodithioate (TLA5-3, MW=250.386 g/mol)

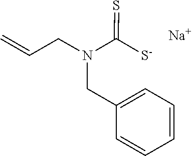

This compound is an analog of TLA5 and was synthesized using a similar procedure.

### Example 3.5.5: Sodium 2,5-dihydro-1H-pyrrole-1-carbodithioate (TLA5-4, MW=173.286 g/mol)

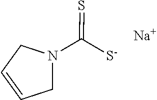

This compound is an analog of TLA5 and was synthesized using a similar procedure.

### Example 3.5.6: Sodium 3,6-dihydropyridine-1(2H)-carbodithioate (TLA5-5, MW=187.312 g/mo)

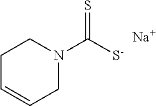

This compound is an analog of TLA5 and was synthesized using a similar procedure.

### Example 3.6: Sodium ((2S,3S)-1-ethoxy-3-methyl-1-oxopentan-2-yl) carbamodithioate (TLA6, LRS01-072)

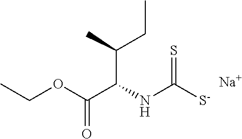

This compound was synthesized similar to the procedure of TLA3. White solid. Yield: 70%.

EXAMPLE 4: The following is a non-limiting example of the present invention. It is to be understood that said example is not intended to limit the present invention in any way. Equivalents or substitutes are within the scope of the present invention.

Example 4.1: A 73-year-old man goes into a clinic complaining of cough, shortness of breath, and chest pain. Once in the examination room, the doctor notes that the man also has a fever. Additionally, as the doctor is listening to the patient's lungs she hears a “crackling” sound. She immediately orders a blood test and a chest x-ray, both come back positive for pneumonia. The doctor prescribes 150 mg of N,N-dimethyldithiocarbamate (DMDC) to be taken orally once a day for a week. She also mentions that the man should get plenty of fluids and rest, and she will see him in a week's time. After the treatment regime, the man returns to the clinic for an evaluation and the man is feeling better. Both a blood test and a chest x-ray confirmed that the man is negative for Streptococcus pneumoniae. No side effects were reported.

Example 4.2: A 35-year-old woman goes to the doctor complaining of chest pain, chills, a cough, fever, and sore throat that has persisted for a week now. The doctor orders a blood test and a chest x-ray, and both come back positive for Valley Fever. The doctor prescribes an inhaler with 50 mg N,N-dimethyldithiocarbamate (DMDC) to be taken twice a day, once in the morning and once at night. The woman is to take 2-3 puffs of the inhaler per dose for a week. After the treatment regime, the woman returns to the doctor for an evaluation. The woman is feeling better and reports that she is no longer experiencing the symptoms that originally brought her to the doctor. Both a blood test and a chest x-ray confirmed that the woman is negative for Coccidioides posadasii. No side effects were reported.

Example 4.3: A father takes his 6-year-old daughter to the doctor's office. She has been complaining of a sore throat, a runny nose, a cough, and difficulty breathing for the last two days. Additionally, the father mentions to the doctor that she has also been running a fever for the past two days. The doctor reviews her symptoms and does a physical examination of the child, before ordering a few laboratory tests. When the tests come back they reveal that the child has an upper respiratory infection caused by Streptococcus pyogenes. The doctor prescribes an inhaler with 25 mg N,N-dimethyldithiocarbamate (DMDC) to be taken twice a day, once in the morning and once at night, for two weeks. The child is to take 2 puffs of the inhaler per dose. The father schedules a follow-up appointment for two weeks later, and takes his daughter home to rest. After the treatment regime, the child returns to the doctor for an evaluation. The child is feeling much better and reports that she is no longer experiencing any symptoms. Follow-up laboratory tests confirm the child is negative for Streptococcus pyogenes. No side effects were reported.

Example 4.4: An 18-year-old man sought medical attention for a skin infection affecting his arms and armpits. The physician conducted a thorough examination, obtaining a small tissue sample and ordering a blood test, both of which confirmed the presence of Methicillin-resistant Staphylococcus aureus (MRSA). The doctor prescribed a topical cream containing 50 mg of N,N-dimethyldithiocarbamate (DMDC), instructing the patient to apply it twice daily, once in the morning and once at night. Upon completing the prescribed treatment regimen, the individual revisited the doctor for a follow-up evaluation. The patient reported a noticeable improvement, expressing relief as the skin rash had completely disappeared. No side effects were reported.

### EMBODIMENTS

The following embodiments are intended to be illustrative only and not to be limiting in any way.

### Embodiment Set A

Embodiment 1A: A method of treating a respiratory infection caused by a pathogenic organism in a patient in need thereof, the method comprising administering a therapeutic amount of N,N-dimethyldithiocarbamate (DMDC) to said patient.

Embodiment 2A: The method of embodiment 1A, wherein the pathogenic organism is a bacteria, a fungus, or a parasite. Embodiment 3A: The method of embodiment 2A, wherein the bacteria is S. pneumoniae. Embodiment 4A: The method of embodiment 2A, wherein the bacteria is S. aureus. Embodiment 5A: The method of embodiment 2A, wherein the fungus is C. posadasii. Embodiment 6A: The method of embodiment 2A, wherein the parasite is a parasitic flatworm. Embodiment 7A: The method of embodiment 6A, wherein the parasitic flatworm is S. mansoni.

Embodiment 8A: The method of embodiment 1A, wherein the respiratory infection is pneumonia. Embodiment 9A: The method of embodiment 1A, wherein the respiratory infection is San Joaquin Valley fever. Embodiment 10A: The method of any one of embodiments 1A-9A, wherein DMDC is administered via inhalation. Embodiment 11A: The method of any one of embodiments 1A-10A, wherein DMDC complexes with copper.

Embodiment 12A: The method of any one of embodiments 1A-11A, wherein DMDC is complexed with copper before being administered to the patient. Embodiment 13A: The method any one of embodiments 1A-12A, wherein DMDC complexes with copper in the patient.

Embodiment 14A: A method of treating a respiratory infection caused by S. pneumoniae in a patient in need thereof, the method comprising administering a therapeutic amount of N,N-dimethyldithiocarbamate (DMDC) to said patient. Embodiment 15A: The method of embodiment 14A, wherein the respiratory infection is pneumonia. Embodiment 16A: The method of embodiment 14A, wherein DMDC is administered via inhalation.

Embodiment 17A: A method of treating pneumonia caused by S. pneumoniae in a patient in need thereof, the method comprising: administering via inhalation a therapeutic amount of N,N-dimethyldithiocarbamate (DMDC) to said patient.

Embodiment 18A: A composition for use in a method of treating a respiratory infection caused by a pathogenic organism, the composition comprising N,N-dimethyldithiocarbamate (DMDC).

Embodiment 19A: The composition of embodiment 18A, wherein the pathogenic organism is a bacteria, a fungus, or a parasite. Embodiment 20A: The composition of embodiment 19A, wherein the bacteria is S. pneumoniae. Embodiment 21A: The composition of embodiment 19A, wherein the bacteria is S. aureus. Embodiment 22A: The composition of embodiment 19A, wherein the fungus is C. posadasii. Embodiment 23A: The composition of embodiment 19A, wherein the parasite is a parasitic flatworm. Embodiment 24A: The composition of embodiment 23A, wherein the parasitic flatworm is S. mansoni.

Embodiment 25A: The composition of embodiment 18, wherein the respiratory infection is pneumonia. Embodiment 26A: The composition of embodiment 18, wherein the respiratory infection is San Joaquin Valley fever. Embodiment 27A: The composition of embodiment 18, wherein the composition is administered via inhalation.

Embodiment 28A: A composition comprising a derivative of N,N-dimethyldithiocarbamate (DMDC):

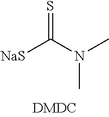

Embodiment 29A: The composition of embodiment 28A, wherein the derivative of DMDC is:

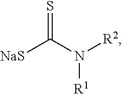

wherein R1 is an alkyl or an aryl group, and R2 is an alkyl or an aryl group.

Embodiment 30A: The composition of embodiment 28A, wherein the derivative of DMDC is:

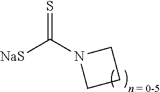

Embodiment 31A: The composition of embodiment 28A, wherein the derivative of DMDC is:

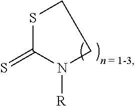

wherein R is an alkyl or an aryl group.

Embodiment 32A: The composition of embodiment 28A, wherein the derivative of DMDC is according to Table 2.

Embodiment 33A.1: A method of treating a respiratory infection caused by a pathogenic organism in a patient in need thereof, the method comprising: administering to said patient a therapeutic amount of a derivative of DMDC according to any one of embodiments 28A-32A. Embodiment 33A.2: A method of treating an infection caused by a pathogenic organism in a patient in need thereof, the method comprising: administering to said patient a therapeutic amount of a derivative of DMDC according to any one of embodiments 28A-32A.

Embodiment 34A: The method of embodiment 33A, wherein the pathogenic organism is a bacterium, a fungus, or a parasite. Embodiment 35A: The method of embodiment 34A, wherein the bacteria is S. pneumoniae or S. aureus. Embodiment 36A: The method of embodiment 34A, wherein the fungus is C. posadasii. Embodiment 37A: The method of embodiment 34A, wherein the parasite is a parasitic flatworm. Embodiment 38A: The method of embodiment 37A, wherein the parasitic flatworm is S. mansoni. Embodiment 39A: The method of embodiment 33A, wherein the respiratory infection is pneumonia. Embodiment 40A: The method of embodiment 33A, wherein the respiratory infection is San Joaquin Valley fever.

Embodiment 41A: The method of embodiment 33A, wherein the derivative of DMDC is administered via inhalation. Embodiment 42A: The method of embodiment 33A, wherein the derivative of DMDC complexes with copper. Embodiment 43A: The method of embodiment 33A, wherein the derivative of DMDC is complex with copper before being administered to the patient. Embodiment 44A: The method of embodiment 33A, wherein the derivative of DMDC complexes with copper in the patient.

Embodiment 45A: A method of treating a respiratory infection caused by S. pneumoniae in a patient in need thereof, the method comprising: administering to said patient a therapeutic amount of a derivative of DMDC according to any one of embodiments 28A-32A. Embodiment 46A: The method of embodiment 45A, wherein the respiratory infection is pneumonia. Embodiment 47A: The method of embodiment 45A, wherein the DMDC derivative is administered via inhalation.

Embodiment 48A: A method of treating pneumonia caused by S. pneumoniae in a patient in need thereof, the method comprising: administering, via inhalation, to said patient a therapeutic amount of a derivative of DMDC according to any one of embodiments 28A-32A.

Embodiment 49A: A composition for use in a method of treating a respiratory infection caused by a pathogenic organism, the composition comprising a derivative of DMDC according to any one of embodiments 28A-32A.

Embodiment 50A: The composition of embodiment 49A, wherein the pathogenic organism is a bacterium, a fungus, or a parasite. Embodiment 51A: The composition of embodiment 50A, wherein the bacteria is S. pneumoniae or S. aureus. Embodiment 52A: The composition of embodiment 50A, wherein the fungus is C. posadasii. Embodiment 53A: The composition of embodiment 50A, wherein the parasite is a parasitic flatworm. Embodiment 54A: The composition of embodiment 53A, wherein the parasitic flatworm is S. mansoni.

Embodiment 55A: The composition of embodiment 49A, wherein the respiratory infection is pneumonia. Embodiment 56A: The composition of embodiment 49A, wherein the respiratory infection is San Joaquin Valley fever. Embodiment 57A: The composition of embodiment 49A, wherein the composition is administered via inhalation.

### Embodiment Set B

Embodiment 1B: A method of treating an infection caused by a pathogenic organism in a patient in need thereof, the method comprising administering a therapeutic amount of a composition comprising N,N-dimethyldithiocarbamate (DMDC) or derivatives thereof to said patient.

Embodiment 2B: The method of embodiment 1B, wherein the pathogenic organism is a bacteria, a fungus, or a parasite.

Embodiment 3B: The method of embodiment 2B, wherein the bacteria comprise Gram positive bacteria. Embodiment 4B: The method of embodiment 2B or embodiment 3B, wherein the bacteria is Streptococcus pneumoniae (S. pneumoniae), pneumococcus, or serotypes thereof (see FIG. 29A-29T); wherein the bacteria is Staphylococcus aureus (S. aureus), or Streptococcus pyogenes (S. pyogenes), Streptococcus anginosus, or Pseudomonas aeruginosa (R aeruginosa). Embodiment 5B: The method of embodiment 2B, wherein the fungus is C. posadasii. Embodiment 6B: The method of any one of embodiments 1B-4B, wherein the infection is a respiratory infection. Embodiment 7B: The method of embodiment 6B, wherein the respiratory infection is pneumonia or San Joaquin Valley fever. Embodiment 8B: The method of any one of embodiments 11B-7B, wherein the composition is administered via inhalation.

Embodiment 9B: The method of embodiment 2B or embodiment 3B, wherein the bacteria is Methicillin-resistant Staphylococcus aureus (MRSA) or Staphylococcus epidermidis. Embodiment 10B: The method of embodiment 9B, wherein the infection is a skin infection. Embodiment 11B: The method of any one of embodiments 1B-10B, wherein the composition is administered topically. Embodiment 12B: The method of embodiment 2B or embodiment 3B, wherein the bacteria Staphylococcus saprophyticus. Embodiment 13B: The method of embodiment 12B, wherein the infection is an urinary tract infection. Embodiment 14B: The method of embodiment 2B, wherein the parasite is a parasitic flatworm, wherein the parasitic flatworm is S. mansoni. Embodiment 15B: The method of embodiment 14B, wherein the infection is Schistosomiasis. Embodiment 16B: The method of embodiment 15B, wherein the composition is administered orally.

Embodiment 17B: The method of any one of embodiments 1B-17B, wherein the derivatives of DMDC are those shown in Table 1. Embodiment 18B: The method of any one of embodiments 1B-17B, wherein the derivative of DMDC is PMDC, BMDC, or AMDC. Embodiment 19B: The method of any one of embodiments 1B-18B, wherein DMDC or derivatives thereof complexes with copper. Embodiment 20B: The method of embodiment 19B, wherein DMDC or derivatives thereof are complexed with copper before being administered to the patient. Embodiment 21B: The method of embodiment 21B, wherein DMDC or derivatives thereof complexes with copper in the patient.

### Embodiment Set C

Embodiment 1C: A composition comprising N,N-dimethyldithiocarbamate (DMDC), or a derivative thereof.

Embodiment 2C: The composition of embodiment 1C, wherein the derivative of DMDC is:

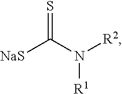

wherein R¹ is an alkyl or an aryl group, and R² is an alkyl or an aryl group.

Embodiment 3C: The composition of embodiment 1C, wherein the derivative of DMDC is:

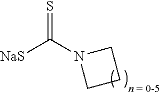

Embodiment 4C: The composition of embodiment 1C, wherein the derivative of DMDC is:

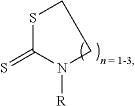

wherein R is an alkyl or an aryl group.

Embodiment 5C: The composition of any one of embodiments 1C-4C, wherein the derivative of DMDC is according to one of the following:

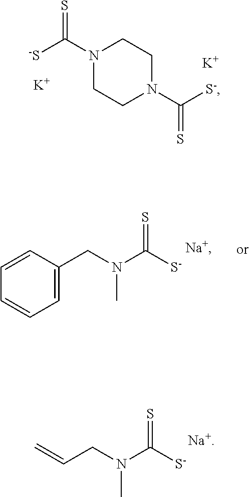

Embodiment 6C: A method of treating an infection caused by a pathogenic organism in a patient in need thereof, the method comprising administering a therapeutic amount of a composition comprising N,N-dimethyldithiocarbamate (DMDC) or derivatives according to any one of embodiments 1C-6C thereof to said patient.

Embodiment 7C: The method of embodiment 6C, wherein the pathogenic organism is a bacteria, a fungus, or a parasite. Embodiment 8C: The method of embodiment 7C, wherein the bacteria comprise Gram positive bacteria. Embodiment 9C: The method of embodiment 7C or embodiment 8C, wherein the bacteria is Streptococcus pneumoniae (S. pneumoniae), pneumococcus, or serotypes thereof (see FIG. 29A-29T); wherein the bacteria is Staphylococcus aureus (S. aureus), or Streptococcus pyogenes (S. pyogenes), Streptococcus anginosus, or Pseudomonas aeruginosa (P. aeruginosa). Embodiment 10C: The method of embodiment 7C, wherein the fungus is C. posadasii. Embodiment 11C: The method of any one of embodiments 6C-10C, wherein the infection is a respiratory infection. Embodiment 12C: The method of embodiment 11C, wherein the respiratory infection is pneumonia or San Joaquin Valley fever. Embodiment 13C: The method of any one of embodiments 6C-12C, wherein the composition is administered via inhalation.

Embodiment 14C: The method of embodiment 7C or embodiment 8C, wherein the bacteria is Methicillin-resistant Staphylococcus aureus (MRSA) or Staphylococcus epidermidis. Embodiment 15C: The method of embodiment 14C, wherein the infection is a skin infection. Embodiment 16C: The method of any one of embodiments 6C-15C wherein the composition is administered topically. Embodiment 17C: The method of embodiment 7C or embodiment 8C, wherein the bacteria Staphylococcus saprophyticus. Embodiment 18C: The method of embodiments 17C, wherein the infection is a urinary tract infection. Embodiment 19C: The method of embodiment 7C, wherein the parasite is a parasitic flatworm, wherein the parasitic flatworm is S. mansoni. Embodiment 20C: The method of embodiment 19C, wherein the infection is Schistosomiasis. Embodiment 21C: The method of claim 20C, wherein the composition is administered orally.

As used herein, the term “about” refers to plus or minus 10% of the referenced number. Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A composition comprising N,N-dimethyldithiocarbamate (DMDC): 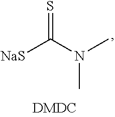
or a derivative thereof.

2. The composition of claim 1, wherein the derivative of DMDC is: 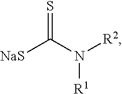
wherein R1 is an alkyl or an aryl group, and R2 is an alkyl or an aryl group.

3. The composition of claim 1, wherein the derivative of DMDC is:

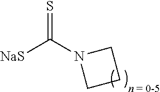

4. The composition of claim 1, wherein the derivative of DMDC is: 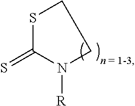
wherein R is an alkyl or an aryl group.

5. The composition of claim 1, wherein the derivative of DMDC is according to one of the following:

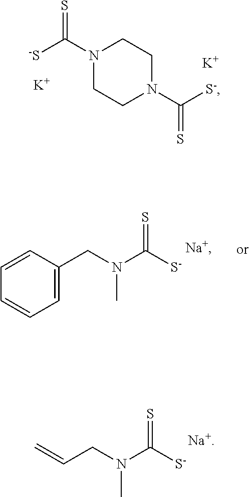

6. A method of treating an infection caused by a pathogenic organism in a patient in need thereof, the method comprising administering a therapeutic amount of a composition comprising N,N-dimethyldithiocarbamate (DMDC) or derivatives according to claim 1 thereof to said patient.

7. The method of claim 6, wherein the pathogenic organism is a bacteria, a fungus, or a parasite.

8. The method of claim 7, wherein the bacteria comprise Gram positive bacteria.

9. The method of claim 7, wherein the bacteria is Streptococcus pneumoniae (S. pneumoniae), pneumococcus, or serotypes thereof; wherein the bacteria is Staphylococcus aureus (S. aureus), or Streptococcus pyogenes (S. pyogenes), Streptococcus anginosus, or Pseudomonas aeruginosa (P. aeruginosa)

10. The method of claim 7, wherein the fungus is C. posadasii.

11. The method of claim 6, wherein the infection is a respiratory infection.

12. The method of claim 11, wherein the respiratory infection is pneumonia or San Joaquin Valley fever.

13. The method of claim 6, wherein the composition is administered via inhalation.

14. The method of claim 7, wherein the bacteria is Methicillin-resistant Staphylococcus aureus (MRSA) or Staphylococcus epidermidis.

15. The method of claim 14, wherein the infection is a skin infection.

16. The method of claim 6, wherein the composition is administered topically.

17. The method of claim 7, wherein the bacteria Staphylococcus saprophyticus.

18. The method of claim 17, wherein the infection is a urinary tract infection.

19. The method of claim 7, wherein the parasite is a parasitic flatworm, wherein the parasitic flatworm is S. mansoni.

20. The method of claim 19, wherein the infection is Schistosomiasis
